(19) United States
(12) Patent Application Publication
(10) Pub. No.: US 2021/0145597 A1
(43) Pub. Date: May 20, 2021

(54) Tissue Engineered Vertebral Discs

(71) Applicants: United States Government As Represented By The Department of Veterans Affairs, Washington, DC (US); The Trustees of the University of Pennsylvania, Philadelphia, PA (US)

(72) Inventors: Sarah E. Gullbrand, Philadelphia, PA (US); Robert L. Mauck, Philadelphia, PA (US); Harvey E. Smith, Philadelphia, PA (US)

(21) Appl. No.: 16/950,335

(22) Filed: Nov. 17, 2020

Related U.S. Application Data
(60) Provisional application No. 62/938,078, filed on Nov. 20, 2019.

(51) Int. Cl. A61F 2/44; A61L 27/12; A61L 27/22; A61L 27/52; A61L 27/26; A61L 27/56

(52) U.S. Cl. CPC A61F 2/442; A61L 27/12; A61L 27/227; A61L 27/52; A61L 2430/38; A61L 27/56; A61F 2310/00796; A61L 2400/12; A61L 27/26

(57) ABSTRACT

Disclosed are engineered vertebral disc implants comprising an engineered vertebral disc, wherein the engineered vertebral disc comprises a nucleus pulposus region and an annulus fibrosus region; and two endplates, wherein the endplates comprise a porous polymer foam, and wherein the endplates comprise channels. Disclosed are methods of treating disc degeneration comprising implanting one or more of the disclosed engineered vertebral disc implants to a subject in need thereof, wherein the endplates of the engineered vertebral disc implant are attached to the vertebra of the subject.

### CROSS-REFERENCE TO RELATED APPLICATIONS

This application claims benefit of U.S. Provisional Application No. 62/938,078, filed Nov. 20, 2019, incorporated by reference herein in its entirety.

### STATEMENT REGARDING FEDERALLY SPONSORED RESEARCH

This invention was made with government support under grants IK2 RX001476, I01 RX002274, IK1 RX002445, and 121 RX003289 awarded by the Department of Veterans Affairs. The government has certain rights in the invention.

### BACKGROUND

Back and neck pain are ubiquitous in modern society, affecting about one half of adults each year, and about two thirds of adults at some point in their lives. Globally, back and neck pain are two of the top four contributors of years lived with disability, and treatment of these conditions has increased healthcare expenditures without evidence of improvement in patient health status. Although the causes of back pain are multifactorial and still not fully understood, degeneration of the intervertebral disc is frequently associated with axial spine pain and neurogenic extremity pain. Intervertebral disc degeneration is characterized by a series of cellular, compositional, and structural changes, including loss of proteoglycan content in the nucleus pulposus (NP), cell death, disorganization of the annulus fibrosus (AF) and a collapse in disc height; together these changes ultimately compromise the mechanical function of the disc. Spinal fusion may be performed in patients with debilitating axial neck or back pain and a severely degenerated intervertebral disc; fusions are also commonly performed when it is necessary to remove the intervertebral disc to restore disc space height (indirectly decompressing the neural foramen) or to gain access to disc-osteophyte complexes that are narrowing the spinal canal. Spinal fusion does not restore native disc structure or mechanical function as it immobilizes the degenerative motion segment; this may contribute to the degeneration of adjacent motion segments due to alterations in whole spine kinematics. Due in large part to the well-recognized clinical problem of adjacent segment degeneration, maintenance of intervertebral disc kinematics after discectomy or decompression of the disc with a mechanical arthroplasty device has emerged as an alternative to fusion procedures, with the goal of restoring disc height while preserving motion. However, the widespread adoption of these devices has been slow, in part due to concerns over subsidence, wear particle generation, and the difficulty of revision surgery.

Considering the social and economic burden of pain and disability associated with intervertebral disc degeneration, and the limitations of currently available surgical treatments, there is a substantial need for new therapies for advanced disc degeneration. Tissue engineering offers great promise—replacement of a degenerative disc with a tissue engineered composite disc has the potential to restore native disc structure, biology, and mechanical function. To date, a number of composite engineered intervertebral discs have been generated, generally involving the combination of a cell-seeded hydrogel (as an analog for the NP region) within a cell-seeded oriented or porous scaffold (as an analog for the AF region). A variety of such composite discs have been characterized in vitro, though few studies have evaluated these constructs in vivo. Understanding the long-term integration and mechanical function of engineered discs in vivo, especially in large animal models at clinically relevant length scales, will be an essential pre-cursor for the translation of these engineered disc technologies into human clinical trials.

### BRIEF SUMMARY

Disclosed are engineered vertebral disc implants comprising an engineered vertebral disc, wherein the engineered vertebral disc comprises a nucleus pulposus region and an annulus fibrosus region; and two endplates, wherein the endplates comprise a porous polymer foam, and wherein the endplates comprise channels.

Disclosed are methods of treating disc degeneration comprising implanting one or more of the disclosed engineered vertebral disc implants to a subject in need thereof, wherein the endplates of the engineered vertebral disc implant are attached to the vertebra of the subject.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows components of an engineered vertebral disc implant: 1) NP hydrogel 2) lamellar PCL scaffold 3) PCL foam endplates.

FIG. 2A and FIG. 2B channels and a keel incorporated into the endplate design (A) and hydroxyapatite (HA) coating to speed boney integration (B).

FIGS. 3A-3J show examples of engineered vertebral disc implant compositions as measured by (A-C) MRI T2 mapping, (D) histology stained for collagens (red) and proteoglycans (blue), and biochemical measurement of (E-G) regional proteoglycan and (H-J) collagen content.

FIGS. 4A-4F show examples of engineered vertebral disc implants compressive (A-C) and tensile (D-F) mechanical properties in the rat tail model.

FIGS. 5A-5D show examples of engineered vertebral disc implants compressive (A-C) mechanical properties and histologic appearance (D) following in vivo implantation for up to 8 weeks in the goat cervical spine. Scale=2 mm.

FIG. 6A and FIG. 6B show a schematic of eDAPS fabrication and cell seeding for the rat and goat models. (A) To fabricate eDAPS sized for the rat caudal disc space, nanofibrous, aligned layered PCL and PEO scaffolds were electrospun, cut into strips at a 30° angle, and rolled around a mandrel to generate the AF region. The AF scaffold was then seeded with bovine AF cells, and a UV curable hyaluronic acid hydrogel was seeded with bovine NP cells. After 2 weeks of culture, the AF and NP regions were combined with the PCL foam endplates to form the eDAPS. (B) To fabricate eDAPS sized for the goat cervical disc space, nanofibrous aligned PCL scaffolds were cut into strips at a 30° angle and seeded with goat MSCs. Goat MSCs were also seeded within an agarose hydrogel to form the NP region. The MSC seeded PCL strips were cultured for 1 week, before rolling strips with opposing fiber angles concentrically using a custom mold to generate the AF region of the eDAPS. After an additional week of culture, the AF and NP regions were combined with the PCL foam endplates to form the eDAPS.

FIGS. 7A-7J show eDAPS structure and composition after in vivo implantation in the rat tail. (A) Representative raw MR images of the first echo of each treatment group (upper), and average T2 maps (lower) of the native disc and eDAPS implants at 10 and 20 weeks (scale=2 mm), obtained at 4.7T. Quantification of eDAPS (B) NP and (C) AF T2 values, bars denote significance (P<0.01). eDAPS biochemical content was further assessed via (D) Alcian blue (proteoglycans) and picrosirius red (collagen) stained histology sections of 10 week and 20 week implants compared with the native rat tail disc space (scale=500 μm). (E) Quantification of GAG content in the NP, (F) AF and (G) PCL endplate regions of the eDAPS. (H) Quantification of collagen content in the NP (P=0.01, 20W vs. pre-implantation), (I) AF (P=0.04, 20W vs. pre-implantation) and (J) PCL endplate regions of the eDAPS (P=0.01, 20W vs. pre-implantation). Quantitative data are shown as mean with standard deviation (n=5-10 per group for MRI data and n=3-4 per group for biochemistry data). Significant differences between groups were assessed with a Kruskal-Wallis with Dunn's multiple comparisons test.

FIG. 8 shows endplate T2 values of rat eDAPS post-implantation. T2 relaxation times within the PCL foam endplates of the eDAPS were (*P<0.05 compared to 10 and 20 week groups) reduced following 10 and 20 weeks implantation.

FIG. 9 shows immunohistochemistry of rat eDAPS after 10 and 20 weeks in vivo. Immunohistochemical staining for type II collagen and chondroitin sulfate was localized to the NP region of the eDAPS, matching the matrix distribution of the native rat tail disc. Type II collagen and chondroitin sulfate were also abundant in the PCL endplate region. Type I collagen was abundant throughout all regions of the eDAPS. Scale=500 μm.

FIG. 10 shows magnified immunohistochemistry of rat eDAPS after 20 weeks in vivo compared to native. Magnified collagen II, chondroitin sulfate and collagen I immunohistochemistry of the NP and AF regions of the 20 week eDAPS implants compared to the native rat tail disc. Scale=250 μm.

FIG. 11 shows hematoxylin and eosin staining of rat eDAPS. Hematoxylin and eosin staining demonstrated maintenance of NP cell distribution in the eDAPS after 10 and 20 weeks in vivo, as well as increased cell infiltration into the AF and endplate regions. Scale=50 μm.

FIG. 12 shows DAPI staining of rat eDAPS. DAPI staining for cell nuclei demonstrated maintenance of cellularity in the NP region of the eDAPS at both time points, in addition to increased cell infiltration of the endplate and AF regions of the eDAPS. Scale=50 μm.

FIGS. 13A-13F show compressive mechanical properties of eDAPS implanted motion segments in the rat tail. (A) Representative stress strain curves of eDAPS prior to implantation, and after 10 and 20 weeks of implantation. The shaded arrow highlights the maturation of mechanical properties towards native values. (B) Quantification of the toe and linear region modulus (P=0.01, 20W toe modulus vs. pre-implantation toe modulus), and (C) transition and maximum strains (*=P<0.01 compared with all groups). Data are shown as mean with standard deviation (n=4-6 per group). Significant differences between groups were assessed with via Kruskal-Wallis with a Dunn's multiple comparison test. (D) μCT scanning before and after the application of physiologic compression in native rat tail motion segments or eDAPS implanted motion segments from the 20-week group. Color scale is representative of bone density. Scale=500 (E) Axial maps of regional disc height generated from the μCT scans via a custom MATLAB code. Color scale indicates local disc height. (F) Compressive strain calculated from the average disc height for the native disc and eDAPS under compression. Data is shown as mean with standard deviation (n=4 per group). Statistical significance between 20W and native strains was assessed via a two-tailed Mann-Whitney test (P=0.11).

FIGS. 14A-14G show in vivo integration of eDAPS in the rat tail. (A) Second Harmonic Generation (SHG) images of the AF-endplate and vertebral body (VB)-endplate in eDAPS implanted for 10 and 20 weeks. The AF-vertebral body interface of the native rat tail IVD is shown for comparison. Scale=200 μm. (B) Mallory-Heidenhain stained histology of native rat tail IVD and the PCL endplate regions at 10 and 20 weeks. Bone matrix stains purple/pink, unmineralized collagen stains blue, and erythrocytes stain orange (arrows). Scale=200 μm. (C) Representative stress strain curves from tension to failure tests of eDAPS implanted motion segments compared to native rat tail motion segments. Two out of three motion segments in the 10-week group had quantifiable tensile properties—the remaining sample failed during dissection (represented as “0” data point on graphs D-E). (D) Quantification of tensile toe and (E) linear region modulus (P=0.03, 10W vs. native) (F) Quantification of failure stress (P=0.01, 10W vs. native) and (G) failure strain (P=0.03, 10W vs. native). Quantitative data are shown as mean with standard deviation (n=3-5 per group). Significant differences between groups were assessed using a Kruskal-Wallis with a Dunn's multiple comparison test.

FIGS. 15A-15-F show eight week quantitative MRI and mechanical properties of eDAPS in a goat cervical disc replacement model. (A) Representative T2-weighted MRIs of eDAPS prior to implantation (scale=2 mm) and (B) 8 weeks post-implantation (arrow, scale=5 mm). (C) Quantification of NP T2 relaxation times in eDAPS implants compared to native goat cervical discs (P=0.04, two-tailed Mann-Whitney test, n=3-13 per group). (D) Representative stress-strain curves from compression testing of goat eDAPS before and after implantation, compared to native goat cervical motion segments. (E) Quantification of toe and linear moduli of eDAPS implanted motion segments compared to native goat cervical motion segments and eDAPS pre-implantation (P=0.02, pre-implantation vs. 8W toe modulus). (F) Quantification of transition and maximum strain in 8 week eDAPS implants compared with native motion segment and eDAPS pre-implantation (P=0.04, 8W vs. pre-implantation transition strain; P=0.03, 8W vs. pre-implantation maximum strain). Quantitative data are shown as mean with standard deviation. Significant differences in mechanical properties between groups (n=3-4 per group) were assessed via a Kruskal-Wallis with Dunn's multiple comparison test.

FIGS. 16A-16E show the translation of eDAPS to a large animal model. Photographs of eDAPS sized for the goat cervical disc space fabricated and seeded with bone marrow derived allogenic MSCs. (A) The C2-C3 disc space was exposed via an anterior approach, and the native disc and portion of the adjacent endplates were removed under distraction. (B) 16 mm diameter by 9 mm high eDAPS, pre-matured for up to 13 weeks, were placed within the prepared disc space and (C) distraction was released. (D) The motion segment was fixed with a cervical fixation plate. (E) All animals recovered from the procedure without complication and retained full cervical spine function.

FIGS. 17A-17D show a four week in vivo performance of eDAPS in a goat cervical disc replacement model. (A) Alcian blue (proteoglycans) and picrosirius red (collagen) stained sections of the eDAPS prior to implantation (after 13 weeks of pre-culture). (B) Alcian blue and picrosirius red stained sagittal histology sections 4 weeks post-implantation. Best and worst representative eDAPS are shown. Scale=1 mm. (C) SHG imaging for organized collagen deposition within the PCL endplate, Scale=200 μm. (D) DAPI staining (scale=50 μm) and immunohistochemistry for collagen II, aggrecan, and collagen I in the NP and AF regions of the eDAPS (scale=250 μm).

FIG. 18 shows a histologic appearance of goat eDAPS implants from all animals. Sagittal Alcian blue and picrosirius red stained histology sections from all four week eDAPS implanted goats (n=4). Scale=1 mm.

FIG. 19A and FIG. 19B show hematoxylin and eosin staining of goat eDAPS. Hematoxylin and eosin staining of eDAPS following 4 weeks implantation in the goat cervical disc space (A) demonstrate the retention of cells within the AF and NP regions, as well as robust cell infiltration into the PCL endplates. Scale=50 (B) Neutrophil infiltration (arrows) into the periphery of the AF indicates a mild inflammatory response. Scale=1 mm (top) and 100 μm (bottom).

FIG. 20 shows immunohistochemistry of goat eDAPS after 4 weeks in vivo. Immunohistochemistry for collagen II, aggrecan and collagen I in 4 week eDAPS implants compared to the native disc. Scale=250 μm.

FIG. 21 shows sagittal μCT slices of eDAPS after 8 weeks in vivo in the goat cervical spine. Mid-sagittal slices from μCT scans of 8 week goat eDAPS implants. Note the PCL endplates were rendered radiopaque with the inclusion of zirconia nanoparticles. Scale=5 mm.

FIG. 22A and FIG. 22B show (A) Alkaline phosphotase activity and (B) Von Kossa staining of HA coated or PCL only foams seeded with bone marrow derived MSCs and cultured for 5 weeks in either Basal or Osteogenic media. *=p<0.05 compared to all other groups. Scale=1 mm.

FIG. 23 shows immunohistochemistry for osteocalcin (scale=1 mm), staining with the Mallory-Heidenhain trichrome stain (pink stain=mineralized collagen, blue=unmineralized collagen, left scale=1 mm, right scale=100 μm), and μCT (scale=1 mm) of acellular PCL and HA coated foams implanted in the rat caudal spine for 10 weeks.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a channel” includes a plurality of such channels, reference to “the engineered vertebral disc” is a reference to one or more engineered vertebral discs and equivalents thereof known to those skilled in the art, and so forth.

“Subject” as used herein refers to a vertebrate. The term “subject” includes domesticated animals (e.g., cats, dogs, etc.), livestock (e.g., cattle, horses, pigs, sheep, goats, etc.), and laboratory animals (e.g., mouse, rabbit, rat, guinea pig, etc.). In one aspect, a subject is a mammal. In another aspect, a subject is a human. The term does not denote a particular age or sex. Thus, adult, child, adolescent and newborn subjects, whether male or female, are intended to be covered.

By “treat” is meant administer or implant one or more of the engineered vertebral disc implants of the invention to a subject, such as a human or other mammal, that has an increased susceptibility for developing disc degeneration, or that has disc degeneration, in order to prevent or delay a worsening of the effects of the disease or condition, or to partially or fully reverse the effects of the disease (e.g. degeneration).

By “prevent” is meant to minimize the chance that a subject who has an increased susceptibility for developing disc degeneration will develop disc degeneration.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Engineered Vertebral Disc Implant

Disclosed are engineered vertebral disc implants. In some aspects, engineered refers to a non-naturally occurring vertebral disc implant.

Disclosed are engineered vertebral disc implants comprising an engineered vertebral disc, wherein the engineered vertebral disc comprises a nucleus pulposus region and an annulus fibrosus region; and two endplates, wherein the endplates comprise a porous polymer foam, and wherein the endplates comprise channels.

In some aspects, the engineered vertebral disc implant can further comprise proteoglycan and collagen. The proteoglycan and collagen can be synthetic or natural. In some aspects, the proteoglycan and collagen are produced by the viable cells present within the engineered vertebral disc.

In some aspects, the depth of the engineered vertebral disc implant measured from the top surface of one endplate to the bottom surface of a second endplate can be 1 mm, 2 mm, 3 mm, 4 mm, 5, mm, 6 mm, 7 mm, 8 mm, 9 mm, 10 mm, 11 mm, 12 mm, 13 mm, 14 mm, 15 mm, 16 mm, 17 mm, 18 mm, 19 mm or 20 mm.

1. Engineered Vertebral Disc

Disclosed are engineered vertebral discs comprising a nucleus pulposus region and an annulus fibrosus region.

i. Nucleus Pulposus Region

The nucleus pulposus is the inner core region of a vertebral disc. In nature and as disclosed herein, the nucleus pulposus region is composed of a gelatinous material.

In some aspects, the nucleus pulposus region comprises a top surface, a bottom surface, and a side edge extending between the top and bottom surfaces and defining a perimeter of the nucleus pulposus region.

In some aspects, the perimeter of the nucleus pulposus is circumferentially surrounded by the annulus fibrosus region. Thus, the nucleus pulposus region is the inner core region and the annulus fibrosus region surrounds it around the perimeter, but not on the top and bottom surfaces.

In some aspects, the nucleus pulposus region comprises a hydrogel. In some aspects, the hydrogel can be, but is not limited to, a hyaluronic acid or agarose hydrogel

In some aspects, the hydrogel comprises viable cells. In some aspects, the viable cells are mesenchymal stem cells or native disc cells, or a combination thereof. In some aspects, the native disc cells can be native nucleus pulposus cells. In some aspects, the viable cells have been cultured prior to adding to the hydrogel. In some aspects, the nucleus pulposus region has been cultured.

ii. Annulus Fibrosus Region

The annulus fibrosus is the exterior of a vertebral disc. The annulus fibrosus surrounds a nucleus pulposus region. The annulus fibrosus can comprise layers or sheets of fibers which keep the gelatinous material of nucleus pulposus from leaking out of the vertebral disc.

In some aspects, the annulus fibrosus region comprises a top surface, a bottom surface, an inner side edge, and an outer side edge that defines a perimeter of the annulus fibrosus region, wherein the inner side edge and the outer side edge extend between the top surface and the bottom surface. In some aspects, the inner side edge can be in contact with the nucleus pulposus region.

In some aspects, the annulus fibrosus region comprises a polymer such as, but not limited to, poly (ε-caprolactone) (PCL), poly(lactic-co-glycolic acid), polylactic acid, poly-DL-lactide, or polydiaxanone. In some aspects, the annulus fibrosus region further comprises polyethylene oxide (PEO). Thus, for example, the annulus fibrosus can be a mixture of PCL and PEO.

In some aspects, the annulus fibrosus region comprises one or more sheets of nano-fibrous polymer. For example, the annulus fibrosus region can comprise one or more sheets of nano-fibrous PCL. In some aspects, the sheets of nano-fibrous polymer (e.g. PCL) can be aligned to form a lamellar structure.

In some aspects, the annulus fibrosus region comprises viable cells. In some aspects, the viable cells are mesenchymal stem cells or native disc cells. In some aspects, the native disc cells are native annulus fibrosus cells.

In some aspects, the cells in the nucleus pulposus region and cells in the annulus fibrosus region are from the same source. In some aspects, the cells in the nucleus pulposus region and cells in the annulus fibrosus region are from different sources. In some aspects, the cells of the nucleus pulposus region or annulus fibrosus region or both have been previously cultured. In some aspects, the annulus fibrosus region has been cultured. In some aspects, the engineered vertebral disc implant has been cultured.

In some aspects, wound concentrically and seeded with either annulus fibrosus cells or mesenchymal stem cells. The nano-fibers within each layer are oriented at a 30 degree angle to the long axis of the implant, and alternate directions (+/−30 degrees) in each successive layer. There could be anywhere from 5-20 layers depending on the size of the implant, and the layer thickness ranges from 200-300 micrometers.

2. Endplates

Disclosed are endplates comprising a porous polymer foam and channels. In some aspects, the endplates can be considered modified endplates since they are not naturally occurring.

In some aspects, the disclosed endplates can have a bone interface side and a vertebral disc interface side. The bone interface side can interact with bone, such as the vertebra. The vertebral disc interface side can interact with a vertebral disc. In some aspects, the disclosed endplates can have a peripheral side edge extending between the bone interface side and the vertebral disc interface side of the endplate.

In some aspects, the vertebral disc interface side of a first endplate is attached to the top surfaces of the nucleus pulposus and annulus fibrosus regions, and wherein the vertebral disc interface side of a second endplate is attached to the bottom surfaces of the nucleus pulposus and annulus fibrosus regions.

In some aspects, each endplate has a thickness less than 5 mm, less than 4 mm, less than 3 mm, less than 2 mm, less than 1 mm. The thickness can be measured from the bone interface side straight through the endplate to the vertebral disc inferface side.

In some aspects, at least one of the two endplates further comprises one or more vascular-promoting agents. In some aspects, the one or more vascular-promoting agents can be, but are not limited to, vascular endothelial growth factor, deferoxamine, nimodipine, or phthalimide neovascularization factor (PNF-1).

i. Polymer Foam

In some aspects, the porous, polymer foam is PCL. In some aspects, the porous, polymer foam can be, but is not limited to, poly(lactic-co-glycolic acid), polylactic acid, poly-DL-lactide, or polydiaxanone.

ii. Hydroxyapatite

Disclosed are engineered vertebral disc implants comprising an engineered vertebral disc, wherein the engineered vertebral disc comprises a nucleus pulposus region and an annulus fibrosus region; and two endplates, wherein the endplates comprise a porous polymer foam, and wherein the endplates comprise channels and wherein the endplates further comprise hydroxyapatite.

In some aspects, the hydroxyapatite can be coated on a surface of the endplates. In some aspects, the hydroxyapatite is present throughout the endplates. In some aspects, the hydroxyapatite can be on the surface and throughout the endplates.

iii. Body and Projection

In some aspects, at least one of the two endplates comprises a body; and a projection on the bone interface side that extends outwardly from the body. The projection can be a variety of shapes and sizes. In some aspects, the projection can be, but is not limited to, circular, rectangular, or triangular. In some aspects, the projection can be 30-75% of the diameter of a endplate along both axes of the endplate. In some aspects, the projection can be up to 80% of the bone interface side of the endplate.

In some aspects, a portion of the body sits directly below the projection.

In some aspects, the projection is centrally positioned on the endplate relative to the transverse axis.

iv. Channels

In some aspects, the one or more of the disclosed endplates comprise channels. In some aspects, the channels are engineered. For example, the channels are not naturally occurring.

In some aspects, the channels of each endplate comprise a plurality of channels that are spaced apart relative to a transverse axis. In some aspects, the plurality of channels of each endplate can be parallel or substantially parallel to one another, and wherein the plurality of channels of each endplate are perpendicular or substantially perpendicular to the transverse axis.

In some aspects, each endplate has a thickness, and wherein each channel of the endplate has a depth that is less than the thickness of the endplate. In some aspects, the depth of each channel can be measured from the bone interface side of the endplate toward the vertebral disc interface side of the endplate.

In some aspects, the channels of each endplate are evenly spaced relative to the transverse axis. In some aspects, sequential channels of each endplate are spaced apart by a distance ranging from 0.5 to 5 mm

In some aspects, each channel of the endplate has opposing ends that are spaced from the peripheral side edge of the endplate.

In some aspects, at a maximum depth of each channel of the endplate, each channel is equally or substantially equally spaced from the vertebral disc interface side of the endplate. In some aspects, at a maximum depth of each channel of the endplate, at least one channel is not equally spaced from the vertebral disc interface side of the endplate.

In some aspects, the projection cooperates with the body to define at least one channel of the endplate. In some aspects, the body solely defines a plurality of channels of the endplate. In some aspects, at least one channel defined by the projection and the body has a depth greater than the depths of the channels defined solely by the body. In some aspects, the depth of each channel defined by the projection and the body has a depth ranging from 0.5 to 3.5 mm, and wherein the depth of each channel defined solely by the body has a depth ranging from 0.5 to 1.5 mm.

In some aspects, the channels are only present in the body of the endplate. In some aspects, the channels are only present in the projection of the endplate. In some aspects, channels are present in both the body and the projection of the endplate.

### C. Methods of Treating Disc Degeneration

Disclosed are methods of treating disc degeneration comprising implanting one or more of the disclosed engineered vertebral disc implants to a subject in need thereof, wherein the endplates of the engineered vertebral disc implant are attached to the vertebra of the subject.

Disclosed are engineered vertebral disc implants for use in treating disc degeneration, wherein the engineered vertebral disc implants are one or more of the engineered vertebral disc implants disclosed herein, wherein the endplates of the engineered vertebral disc implant are attached to a vertebra of a subject with disc degeneration.

Disclosed are engineered vertebral disc implants for use in treating disc degeneration, wherein the engineered vertebral disc implant comprises an engineered vertebral disc, wherein the engineered vertebral disc comprises a nucleus pulposus region and an annulus fibrosus region; and two endplates, wherein the endplates comprise a porous polymer foam, and wherein the endplates comprise channels. In some aspects the engineered vertebral disc implant of claim 1 wherein the endplates further comprise hydroxyapatite. In some aspects, at least one of the two endplates of the engineered vertebral disc implant comprises: a body; and a projection on the bone interface side that extends outwardly from the body.

In some aspects, the engineered vertebral disc implant can be cultured prior to implanting to a patient. In some aspects, the viable cells within the engineered vertebral disc implant have been cultured prior to implanting to a patient. In some aspects, the engineered vertebral disc implant can be cultured in the presence of TGF-β3 prior to implanting to a patient. In some aspects, the culturing results in differentiation of the cells in the engineered vertebral disc implant. The differentiation of cells can lead to the engineered vertebral disc implant having properties similar to that of a natural vertebral disc. In some aspects, the properties obtained by culturing can include, but are not limited to, maintenance of cell viability, accumulation of collagen (types I and II) and proteoglycan matrix within the nucleus pulposus and annulus fibrosus regions, integration of the annulus fibrosus and nucleus pulposus regions, and maturation of compressive mechanical properties towards native levels.

In some aspects, the disclosed methods can further comprise removing the degenerated disc prior to implanting the engineered vertebral disc implant.

In some aspects, the disclosed methods can further comprise removing a portion of a vertebra prior to implanting engineered vertebral disc implants. In some aspects, the vertebra in which a portion can be removed is a vertebra directly above or below where the engineered vertebral disc implant will be implanted. In some aspects, removing a portion of the vertebra comprises scraping or drilling into the vertebra. In some aspects, removing a portion of the vertebra can allow for blood and bone cells to integrate into the engineered vertebral disc implant.

In some aspects, the cells in the engineered vertebral disc implant can be cells obtained from the subject. For example, cells can be obtained from a subject, deposited on or administered to a scaffold, such as the engineered vertebral disc or a portion thereof, used to create an engineered vertebral disc implant. In some aspects, the scaffold comprising the cells are cultured allowing for the cells to differentiate prior to implanting the engineered vertebral disc into a subject. In some aspects, the subject from which the cells are obtained is the same subject in the engineered vertebral disc implant is implanted. In some aspects, the subject from which the cells are obtained is a different subject than the subject in which the engineered vertebral disc implant is implanted.

In some aspects, the endplates can be attached to the vertebra via the bone interface side. In some aspects, a first endplate is attached to the vertebra above it and a second endplate is attached to the vertebra below it. The attachment of the endplate to the vertebra can be via a variety of mechanisms, for example, placement of screws, with our without additional hardware including plates or buttresses. In some aspects, the hardware can be made of metal or bio-resorbable polymers. In some aspects, biodegradable polymers can include, but are not limited to, the Poly (α-hydroxy acids) class—such as poly (lactic acid), poly (glycolic acid) and poly (lactic-co-glycolide). In some aspects, the endplates integrate with the vertebra. In some aspects, prior to and during attachment of the endplate to the vertebra, proper alignment of the endplate with the vertebrate is performed. Proper alignment would be understood by those of skill in the art. In some aspects, proper alignment can mean once the engineered vertebral disc implant is attached, the vertebra on top of the implant and below the implant are aligned in a similar manner to a healthy spine.

In some aspects, the subject's cells infiltrate into the engineered vertebral disc implant. The infiltration of the subject's cells into the engineered vertebral disc implant can result in viable cells in the endplates of the engineered vertebral disc implant.

In some aspects, the endplates comprise collagen. In some aspects, the endplates vascularize. The vascularization can occur from the blood and cells infiltrating from the surrounding bone and tissue.

### D. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more components of the engineered vertebral disc implant. Disclosed are kits comprising one or more of the disclosed nucleus pulposus regions, one or more of the disclosed annulus fibrosus regions, one or more of the disclosed engineered vertebral discs, one or more of the disclosed endplates, and/or cells.

### EXAMPLES

### A. Example 1

1. Background

A variety of tissue engineered discs have been described in the literature, with engineered subcomponents to mimic the inner nucleus pulposus (NP, water and proteoglycan rich) and outer annulus fibrosus (AF, lamellar collagen structure) substructures of the native disc. The only other engineered disc construct aside from our design to have been evaluated in vivo is composed of a cell-seeded alginate hydrogel for the NP region and a collagen gel for the AF region which has been compacted and circumferentially aligned by the cells seeded within it. The disclosed invention is different than prior technology in that it utilizes a lamellar structure for the AF region which more closely recapitulates the native tissue. Biomaterial interfaces attached to the NP and AF components are also included which promote integration with the native bone.

2. Description

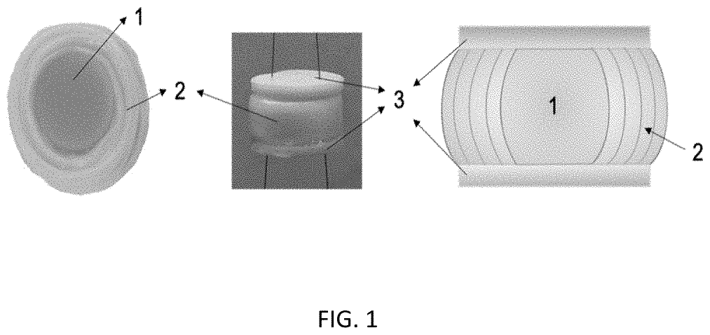

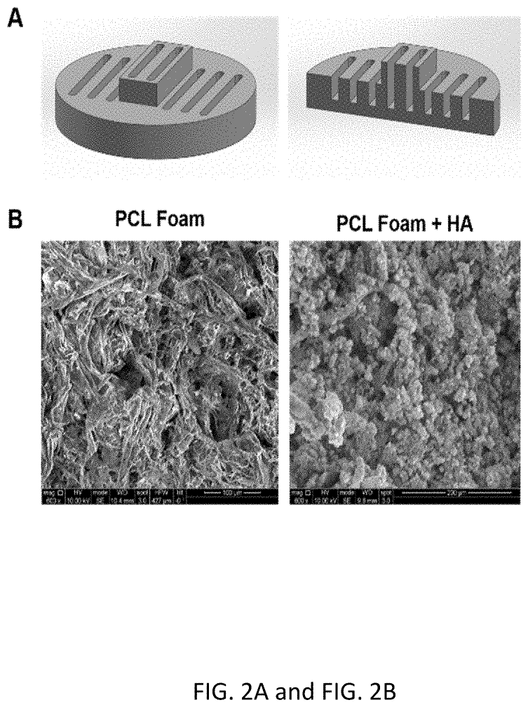

Disclosed herein is an endplate-modified disc-like angle-ply structure (eDAPS), also referred to throughout as an engineered vertebral disc implant, which is composed of four components: (FIG. 1) Hydrogel: A hydrogel, seeded with cells (mesenchymal stem cells, or native disc cells) comprises the nucleus pulposus region of the engineered disc. Aligned, nano-fibrous, lamellar poly (ε-caprolactone): The annulus fibrosus region of the disc is fabricated from sheets of aligned, electrospun poly (ε-caprolactone) (PCL). The sheets of nanofibers are cut into strips such that the fibers are oriented at a 30° angle in each strip. The strips are seeded with cells (mesenchymal stem cells, or native disc cells) and then coupled to achieve opposing ±30° fiber orientation in sequential layers, and wound in a circular mold to create a lamellar structure. The cell seeded hydrogel is then placed in the center of the annulus structure to create a composite AF-NP structure. Porous poly (ε-caprolactone) foams: Two porous PCL foams are fabricated via a salt leaching process, and are attached to both sides of the AF-NP composite during culture. These “endplate” regions are designed to interface with the adjacent bone following in vivo implantation and provide an interface for integration to occur. The foams can also contain channels and can be modified with hydroxyapatite to promote boney integration (FIG. 2). Microspheres containing vascular-promoting agents can be incorporated at the edge of the foam that will interface with the native bone.

The eDAPS can be fabricated at multiple length scales, including that sized for the human cervical disc space. At this large size scale, eDAPS seeded with cells are pre-cultured for up to 17 weeks in chemically defined media with TGF-β3, to allow the mechanical properties of the implant to mature as the cells deposit a progteoglycan and collagen rich matrix within the scaffold.

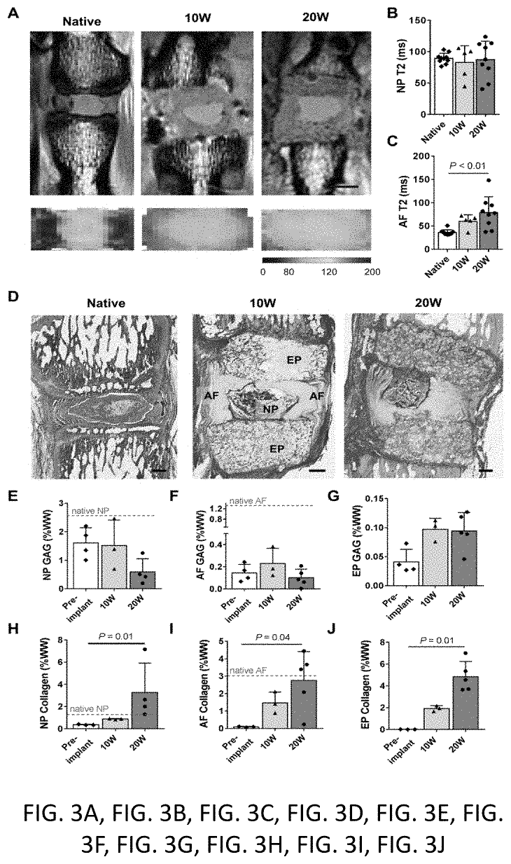

The performance of the eDAPS has been evaluated in vivo in both small (rat tail) and large (goat cervical spine) animal models. Following implantation in vivo in a rat tail disc replacement model, the MRI, histology, and biochemistry data demonstrate that the eDAPS composition and hydration are stable over a 20 week period in vivo, and that the eDAPS recapitulate many of the hallmarks of native disc composition and structure (FIG. 3). Evidence of maturation of the endplate-vertebral body interface was observed from 10 to 20 weeks implantation. Increased cell infiltration into the AF and endplate regions was evident on histology samples from 10 to 20 weeks, while cells remained within the NP over the same time period. Given that the engineered endplates were acellular at the time of implantation, this indicates that native cells from the adjacent tissues were able to migrate into the open porous structure of the endplate over time and produce matrix.

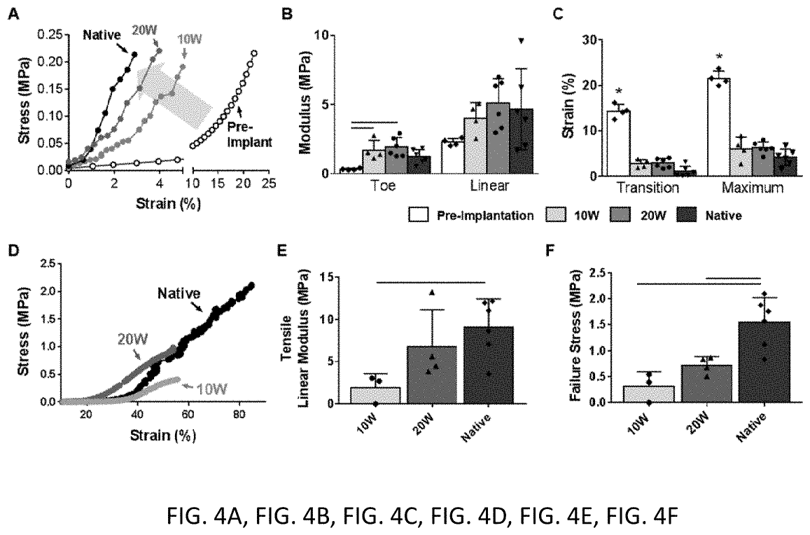

Marked maturation of eDAPS compressive mechanical properties (FIGS. 4A-C) was also observed with increasing duration of in vivo implantation, ultimately matching native motion segment values in most aspects. The eDAPS toe region modulus significantly increased after 20 weeks compared to pre-implantation values, and was not different from the native disc toe region modulus at either 10 or 20 weeks. Toe region mechanics in the disc are largely dictated by the function of the NP, indicating that the NP region continues to mature after in vivo implantation, contributing to overall disc function. The linear region modulus was not significantly affected by in vivo implantation, though there was an increasing trend compared to pre-implantation levels, and implanted eDAPS were not different from the native disc at 10 or 20 weeks in terms of linear region compressive modulus. The linear region response of the eDAPS is initially dominated by the PCL comprising the AF region of the eDAPS; as such, native linear region mechanics are recapitulated to some extent even prior to implantation. Integration strength of the eDAPS with the native tissue was also assessed in the rat tail model via tension to failure testing (FIGS. 4D-F). Increases in the tensile toe region modulus and linear region modulus were evident from 10 to 20 weeks implantation. The toe and linear region moduli in tension were within the range of the native rat tail in the 20 week eDAPS implanted motion segments. Failure stress and strain of the eDAPS were 46.6% and 50.1% of native values after 20 weeks in vivo, respectively.

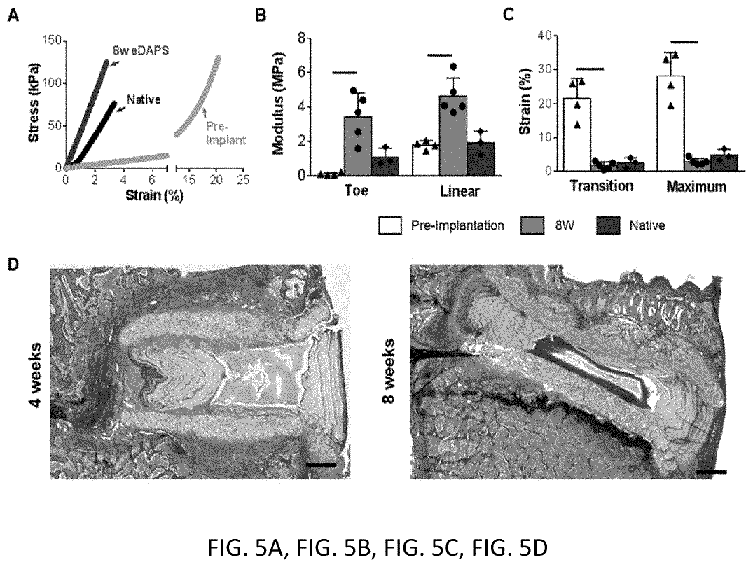

Next, the eDAPS in vivo performance were evaluated in a more clinically relevant model, and the goat cervical spine was chosen due to its semi-upright nature, and similarities in disc height and area to the human cervical spine. After implantation of pre-cultured eDAPS in the goat cervical spine (FIG. 5), eDAPS composition and structure were maintained at or above pre-implantation levels after 4 weeks in vivo. After 8 weeks in vivo, there was an increase in collagen matrix deposition within the PCL endplates and the annulus fibrosus, accompanied by slight reductions in proteoglycan staining within the NP region compared to 4 weeks. SHG images also revealed the deposition of organized collagen within the initially acellular PCL endplates, resulting in nascent integration of the eDAPS with the vertebral bodies at 4 weeks that further matured after 8 weeks. Compressive mechanical testing showed significant maturation of eDAPS mechanical properties from pre-implantation values after 8 weeks in vivo. While toe and linear region moduli of the eDAPS implanted motion segments trended higher than native goat cervical disc moduli, the transition and maximal strains were significantly reduced from pre-implantation levels at 8 weeks, and were not significantly different from the native cervical motion segment.

### B. Example 2

1. Introduction

To address the current problems with engineered discs, endplate-modified disc-like angle ply structures (eDAPS), also referred to as engineered vertebral disc implants, composed of three distinct components were developed to mimic the hierarchical structure of the native spinal motion segment. The NP region is formed from a cell-seeded hyaluronic acid or agarose hydrogel, whereas the AF region is composed of cell-seeded, concentric layers of aligned, nanofibrous poly(ε-caprolactone) (PCL). Hydrogels were selected for the NP region to recapitulate the highly hydrated state of the native NP, whereas PCL was selected for the AF region due to its slow degradation rate, robust mechanical properties, and its ability to be fabricated via electrospinning into ordered structures that replicate the fiber architecture of the annulus fibrosus. The AF and NP regions are combined with two acellular, porous PCL foams as endplate (EP) analogs to generate the eDAPS construct. These eDAPS have previously been evaluated in a rat caudal disc replacement model in short term studies, with endplate-modified constructs outperforming those without endplates. Herein, the long-term in vivo integration and mechanical function of eDAPS is demonstrated in the rat caudal spine. These engineered discs maintained composition and structure while functionally maturing in vivo, reaching near native tensile and compressive mechanical properties by 20 weeks. To further advance the clinical translation of tissue engineered disc replacements, human-sized eDAPS were successfully implanted into the cervical spine of a large animal (caprine) model.

2. Results

i. eDAPS Structure and Composition are Maintained In Vivo.

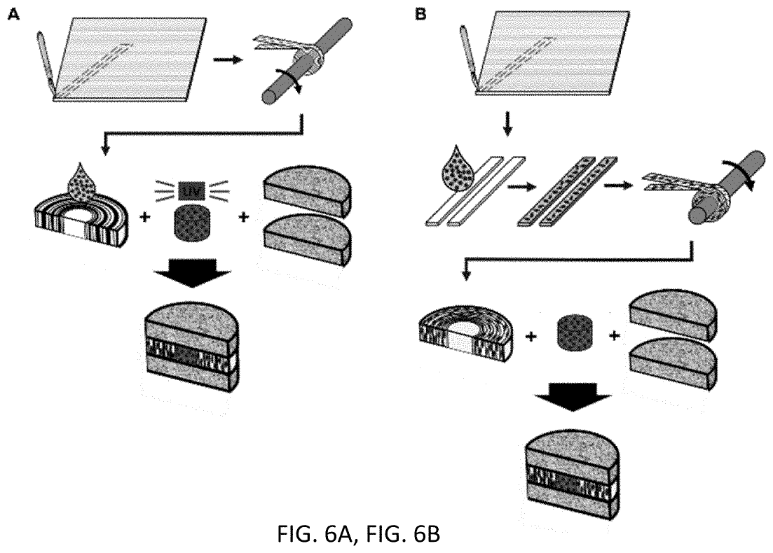

To determine whether a tissue engineered disc can recapitulate the structure and function of the native disc with long-term implantation, eDAPS were implanted in vivo in a small animal disc replacement model for up to 20 weeks. eDAPS sized for the rat caudal spine (4-5 mm diameter, 5-6 mm high) were fabricated, seeded with bovine NP cells within a hyaluronic acid hydrogel and bovine AF cells within a layered PCL/poly(ethylene oxide) (PEO) scaffold, and combined with two acellular PCL foam endplates (FIG. 6).) eDAPS were cultured for 5 weeks in vitro in chemically defined media with TGF-β3 prior to implantation in the athymic rat caudal disc space for either 10 weeks (n=5) or 20 weeks (n=9) with external fixation to immobilize the motion segment and ensure eDAPS retention.

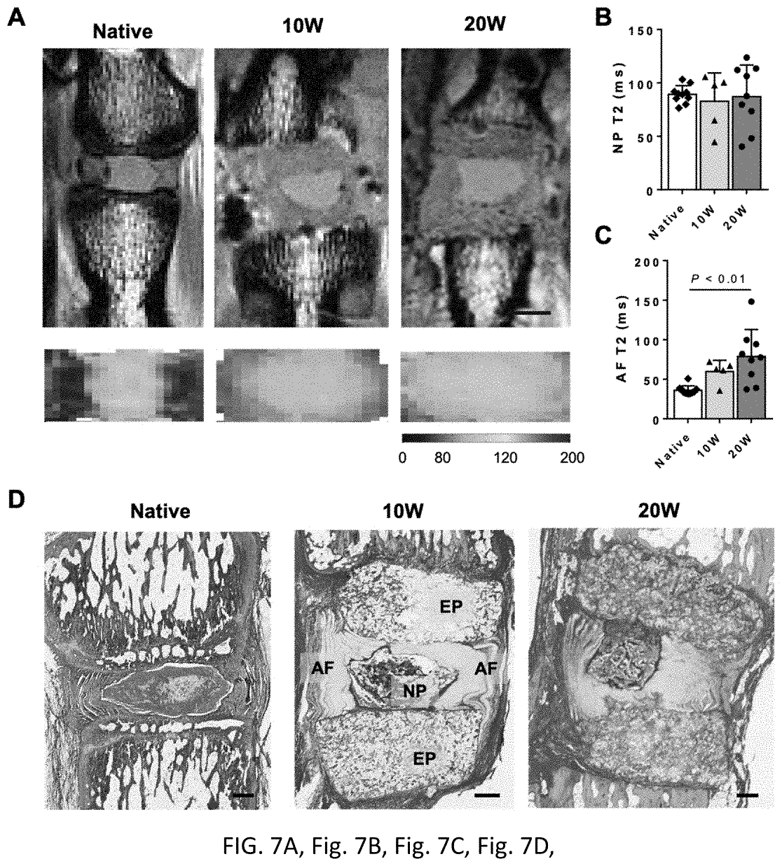

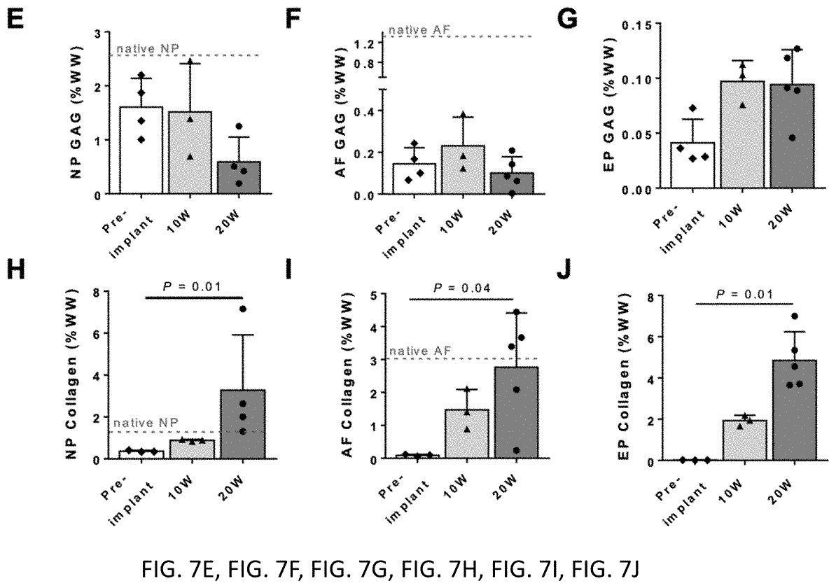

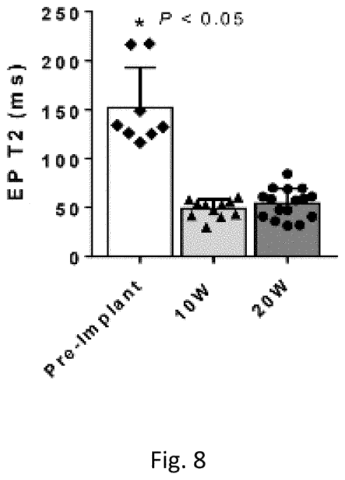

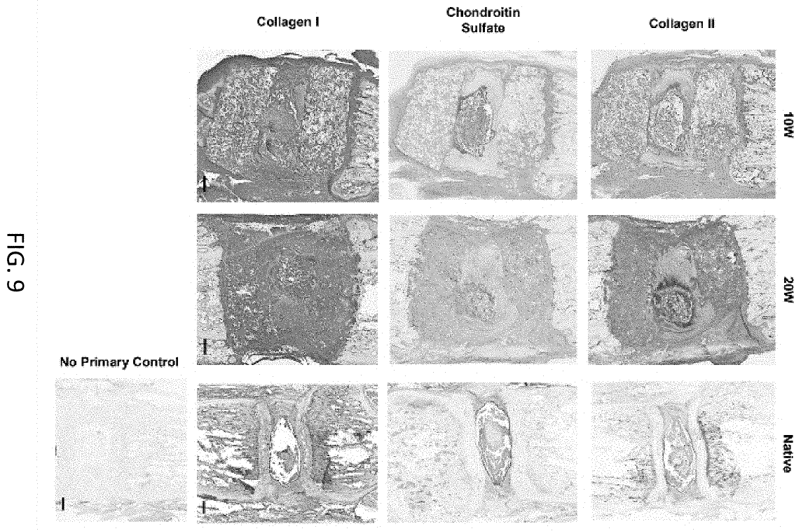

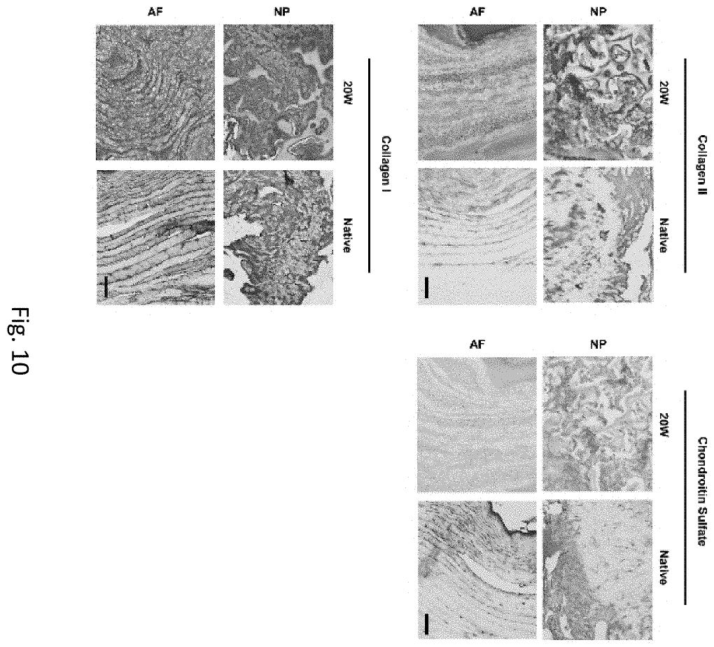

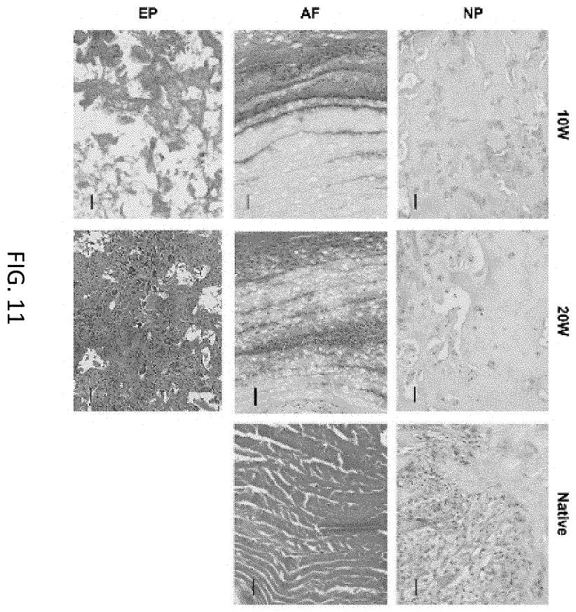

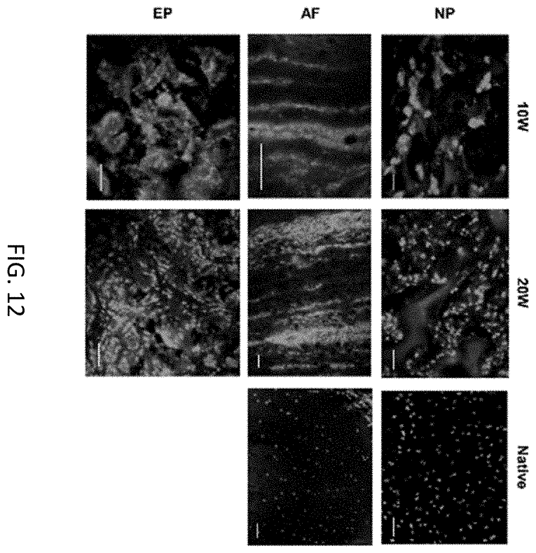

Magnetic resonance imaging (MRI), particularly T2-weighted MRI, is a clinical tool commonly used to assess disc health. Quantitative T2 mapping of the disc has also demonstrated that T2 relaxation times in the NP are positively correlated with disc hydration, proteoglycan content, and mechanics. T2 mapping (FIG. 7A) of implanted eDAPS demonstrated that T2 relaxation times in the NP were maintained at native values after 10 or 20 weeks of in vivo implantation (FIG. 7B). eDAPS AF T2 values, however, were significantly higher (P<0.01) than the native AF at 20 weeks (FIG. 7C). Conversely, endplate T2 values decreased from pre-implantation values at 10 and 20 weeks, indicative of new matrix deposition in this region (FIG. 8). Overall, this MRI data indicated that the eDAPS maintained their biochemical composition and hydration within the NP and AF with long-term implantation.

MRI results were confirmed via histology and quantitative biochemistry. Alcian blue and picrosirius red stained sections of eDAPS implanted motion segments showed strong and persistent proteoglycan staining in the NP and increasing collagen deposition in the AF from 10 to 20 weeks—recapitulating the matrix distribution of the native disc (FIG. 7D). Evidence of increased integration of the engineered endplate with the native vertebral bodies was also observed with longer durations of implantation. In the native disc, type II collagen and chondroitin sulfate are distributed predominantly within the NP region, with little expression in the AF, which is rich in type I collagen. This distribution of matrix is critical for the mechanical function of the disc—the hydrostatic pressure generated in the proteoglycan-rich and highly hydrated NP places the AF in tension, allowing the disc to bear compressive loads. Immunohistochemistry (FIG. 9, FIG. 10) revealed similar patterns of matrix distribution within the eDAPS after 10 and 20 weeks of implantation, with robust staining for type II collagen and chondroitin sulfate in the NP region. Type II collagen and chondroitin sulfate staining was lower in the AF region, but was present in the PCL foam endplates, and increased from 10 to 20 weeks. Type I collagen was evenly distributed throughout the eDAPS at both the 10 and 20 week time points.

In accordance with histologic findings, NP, AF, and endplate glycosaminoglycan (GAG) content remained at pre-implantation values over 20 weeks post-implantation (FIGS. 7E-G). NP, AF, and endplate collagen content significantly increased (P=0.01, 0.04 and 0.01, respectively) from pre-implantation values after 20 weeks in vivo (FIGS. 7H-J). NP and AF GAG and collagen content were generally in the range of the native rat tail NP and AF, with the exception of AF GAG content, which remained below native values at both time points.

These MRI, histology, and biochemistry data demonstrate that the eDAPS composition and hydration are stable over a 20 week period in vivo, and that the eDAPS recapitulate many of the hallmarks of native disc composition and structure. Evidence of maturation of the endplate-vertebral body interface was observed from 10 to 20 weeks implantation. Increased cell infiltration into the AF and endplate regions was evident on histology samples from 10 to 20 weeks, while cells remained within the NP over the same time period (FIG. 11, FIG. 12). Given that the engineered endplates were acellular at the time of implantation, this indicates that native cells from the adjacent tissues were able to migrate into the open porous structure of the endplate over time and produce matrix.

ii. eDAPS Mechanical Properties Approach Native Values In Vivo.

To elucidate how the observed integration and maturation of the eDAPS in vivo affected spine mechanical function, the compressive and tensile properties of eDAPS implanted motion segments were quantified. After 10 and 20 weeks in vivo, vertebra-eDAPS-vertebra motion segments were isolated and subjected to compressive mechanical testing under physiologic loading (20 cycles compression, from 0 to −3N ˜0 to 0.25 MPa). This loading regime represents the application of 0.5 times human body weight stress to the engineered disc, the most demanding mechanical testing profile considered to date for any in vivo study of engineered disc implantation, and 16-fold greater than previously used to characterize the mechanical function of tissue engineered discs after implantation in the rat caudal spine. From these tests, the compressive mechanical properties of the eDAPS implanted motion segments were compared to native rat tail motion segments, as well as the properties of the eDAPS construct after 5 weeks of in vitro culture (pre-implantation).

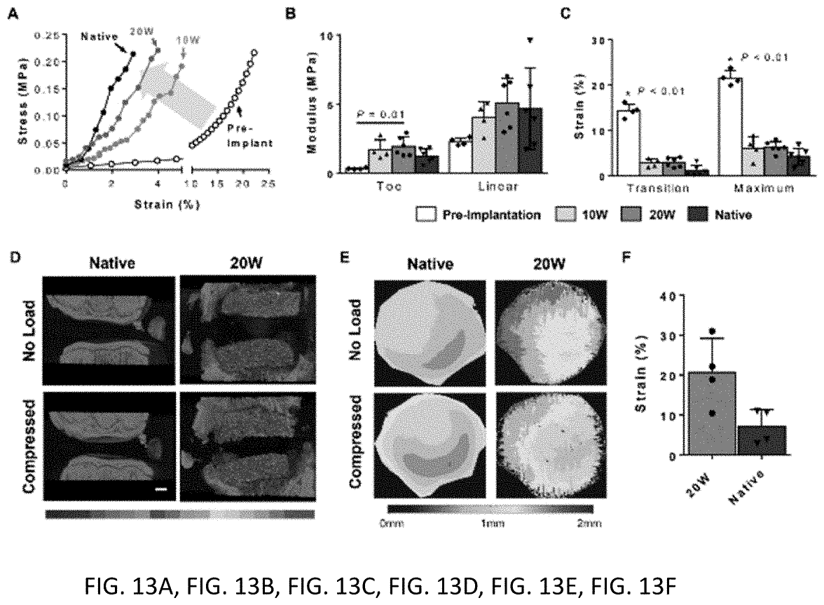

Marked maturation of eDAPS compressive mechanical properties was observed with increasing duration of in vivo implantation, ultimately matching native motion segment values in most aspects (FIG. 13A). The eDAPS toe region modulus significantly increased (P=0.01) after 20 weeks compared to pre-implantation values, and was not different from the native disc toe region modulus at either 10 or 20 weeks (FIG. 13B). Toe region mechanics in the disc are largely dictated by the function of the NP, suggesting that the NP region continues to mature after in vivo implantation, contributing to overall disc function. The linear region modulus was not significantly affected by in vivo implantation, though there was an increasing trend compared to pre-implantation levels, and implanted eDAPS were not different from the native disc at 10 or 20 weeks in terms of linear region compressive modulus (FIG. 13B). The linear region response of the eDAPS is initially dominated by the PCL comprising the AF region of the eDAPS; as such, native linear region mechanics are recapitulated to some extent even prior to implantation. From histology, it was evident that the PCL within the eDAPS AF persisted over 20 weeks in vivo (FIG. 7D), and therefore likely still contributed to the linear region mechanics at that time point, as new tissue was deposited and accumulated in this region. The eDAPS construct is in an immature state prior to implantation, with low levels of matrix, and the transition and maximum strains of the construct are initially super-physiologic. However, after 10 or 20 weeks in vivo, both transition and maximum strains were significantly reduced (P<0.01) to native values, indicative of the compositional maturation of the construct and integration with the native tissue (FIG. 13C). Overall, these results demonstrate that eDAPS recapitulate native motion segment mechanical function after long-term implantation, and can withstand the demanding loading environment of the spinal motion segment.

Macroscopic compression testing provides information on the mechanical function of the eDAPS as a whole; thus, the function of the disc region itself (tissue located between the endplates) cannot be determined from this method. As the engineered endplates integrate with the native vertebral body and remodel over time into bone, the engineered disc (DAPS) region will be increasingly responsible for the function of the motion segment. To resolve the mechanical properties of the DAPS, independent of the endplates, mechanics were assessed after 20 weeks in vivo, using a micro-computed tomography (μCT) coupled compression test. For the 20-week implantation group, endplates were rendered radiopaque via the inclusion of zirconia oxide nanoparticles, allowing for μCT visualization of the disc/DAPS boundary. After macroscopic compression testing, vertebra-eDAPS-vertebra motion segments and native rat tail motion segments were subjected to μCT scans before and after the application of a 3N compressive load, representing 0.5 times body weight (FIG. 13D). The height of the engineered disc (DAPS) between the radiopaque PCL endplates and the height of the native disc between vertebral endplates was quantified from pre- and post-compression three dimensional μCT renderings. This analysis enabled computation of strain across the disc itself. Spatial maps of axial disc height (FIG. 13E) revealed similar distributions in disc height across the native disc and DAPS after compression, though the initial DAPS height was greater than native values. Compressive strain within the DAPS under physiologic compression trended (P=0.11) higher than the native disc (FIG. 13F). This indicates that, although the macroscopic properties of the eDAPS as a whole recapitulate those of the native motion segment when measured in a dynamic setting, some mechanical insufficiency remains in the disc region of the implant at 20 weeks when measured at equilibrium. This can be due to deficiencies in eDAPS GAG content compared with the native disc, particularly in the AF region.

iii. eDAPS Functionally Integrate with the Native Tissue.

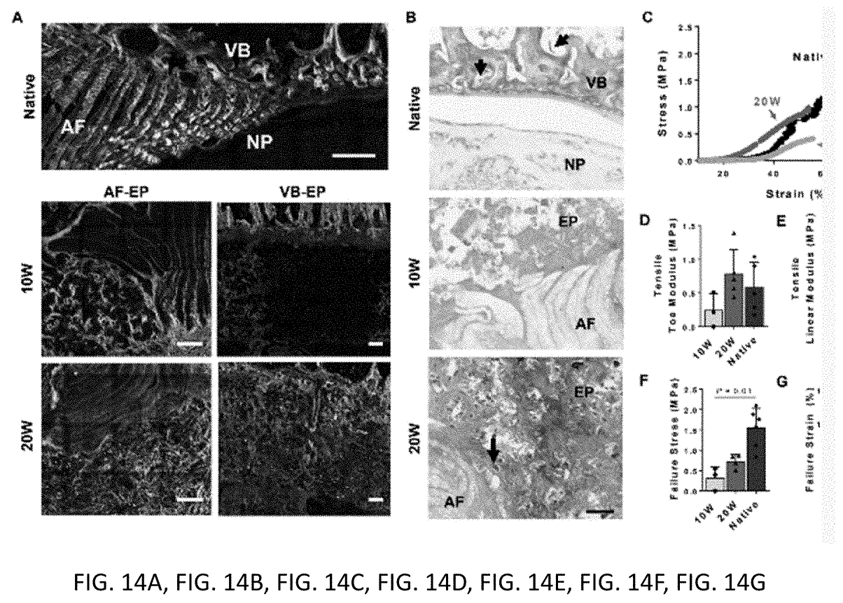

Histology, biochemical content and macroscale mechanics indicated progressive integration of the eDAPS with the native tissue after implantation. The extent of this integration was further assessed via second harmonic generation (SHG) imaging, which provides visualization of organized collagen within tissue. SHG signal within the engineered endplates increased substantially from 10 to 20 weeks (FIG. 14A). SHG also demonstrated increasingly robust integration of the eDAPS at both the AF-endplate and endplate-vertebral body interfaces with increasing time post-implantation. Mineralized collagen and sparse vascularization was evident in the engineered endplates at 20 weeks, as observed via Mallory-Heidenhain stained histology sections (FIG. 14B), in which bone matrix stains dark gray, unmineralized collagen stains light grey, and erythrocytes stain shown with arrows.

This progressive integration resulted in tangible changes in tensile mechanical properties, which improved from 10 to 20 weeks implantation (FIG. 14C). After compressive macro- and micro-CT based mechanical testing, a complete release of the soft tissue surrounding the eDAPS implants was performed. At 10 weeks, the act of freeing the motion segment from the surrounding soft tissue resulted in failure in one out of three samples. Conversely, all samples in the 20-week group remained intact after circumferential tissue release. When these 20-week eDAPS implanted motion segments were tested to failure in tension, failure occurred at the AF/NP-PCL endplate junction in all samples. In the native rat tail, tensile failure occurred at the growth plate. Increases in the tensile toe region modulus and linear region modulus were evident from 10 to 20 weeks implantation. The toe and linear region moduli (FIGS. 14D-E) in tension were within the range of the native rat tail in the 20 week eDAPS implanted motion segments. Failure stress and strain (FIGS. 14F-G) of the eDAPS were 46.6% and 50.1% of native values after 20 weeks in vivo, respectively. Tensile properties to failure of a tissue engineered disc after in vivo implantation have not been previously reported. The tensile stresses reached in this study (applying tension to failure) are 45-fold higher than previously reported (675 kPa vs 15 kPa) during non-destructive tensile testing (±3% applied tensile strain) of a tissue engineered disc implanted in the rat caudal disc space.

iv. eDAPS Compositionally and Functionally Mature after Implantation in a Large Animal Model.

The results in the rat tail disc replacement model were promising, but rat tail discs are a fraction of the size of a human lumbar or cervical disc, and the rat caudal spine also has a different anatomy and mechanical loading environment compared with the human spine. Thus, clinical translation of the eDAPS requires scale up of the constructs in size and evaluation in a large animal model with comparable geometry and mechanical function to the human spine. The human cervical spine is a likely first clinical target for a tissue engineered total disc replacement, given that metal and plastic artificial total disc implants have already been used in this location with some success, and it has a smaller size and less demanding mechanical loading environment compared to the lumbar spine. The goat cervical spine was chosen as the large animal model in which to next evaluate eDAPS performance. The goat is a commonly used large animal model for spine research, and the goat cervical spine has the benefit of semi-upright stature and disc dimensions similar to the human cervical spine. The feasibility of the scale up of DAPS to large, clinically relevant size scales has been demonstrated, and DAPS sized for the goat cervical disc space compositionally and functionally mature during in vitro culture were illustrated, albeit at a slower rate than smaller DAPS.

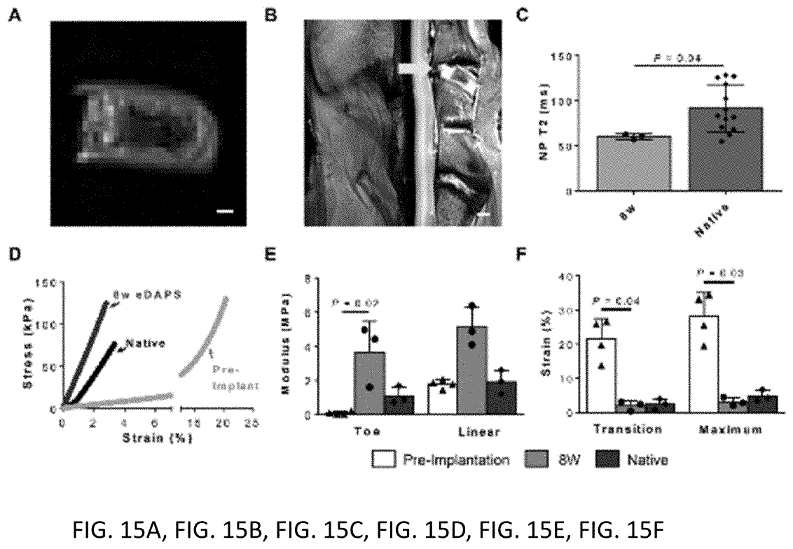

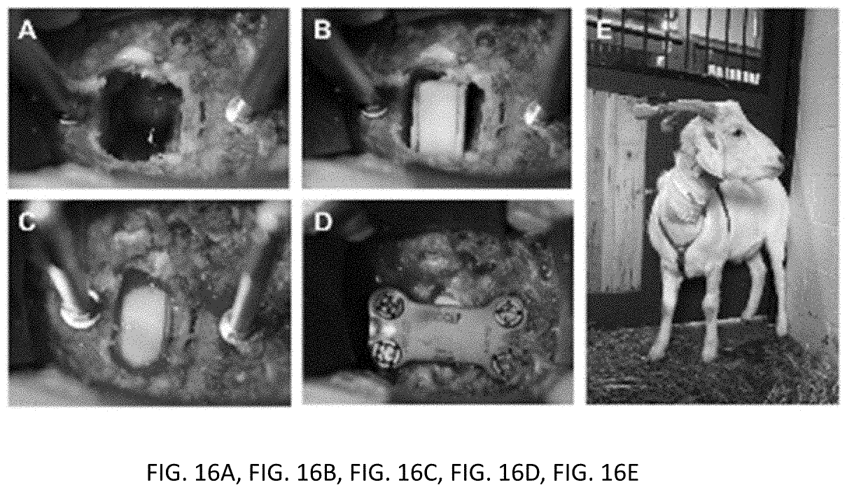

To evaluate the eDAPS in this context, constructs sized for implantation in the goat cervical spine (9 mm high, 16 mm diameter) were fabricated using an agarose hydrogel for the NP region and concentric layers of aligned PCL for the AF region, combined with acellular PCL foam endplates (FIG. 15). To use a more translationally relevant cell source for the large animal studies, eDAPS were seeded with allogeneic goat bone-marrow derived mesenchymal stem cells (MSCs) and cultured for 13-15 weeks prior to implantation. The C2-C3 disc space of 7 male, large frame goats was exposed and the native disc and portion of the adjacent vertebral boney and cartilaginous endplate were removed under distraction, using tools commonly used in human cervical spine surgery. The eDAPS was placed within the evacuated space, distraction was released (placing the eDAPS under compression), and the interspace was immobilized with an anterior cervical plate (FIG. 16A-D). Plate fixation was utilized as previous work demonstrated issues with engineered disc retention in the beagle cervical spine without fixation. All goats recovered from the surgical procedure without complication (FIG. 16E), and maintained full cervical spine function. Four weeks post-implantation, four animals were euthanized and the cervical spines were harvested for histologic analyses.

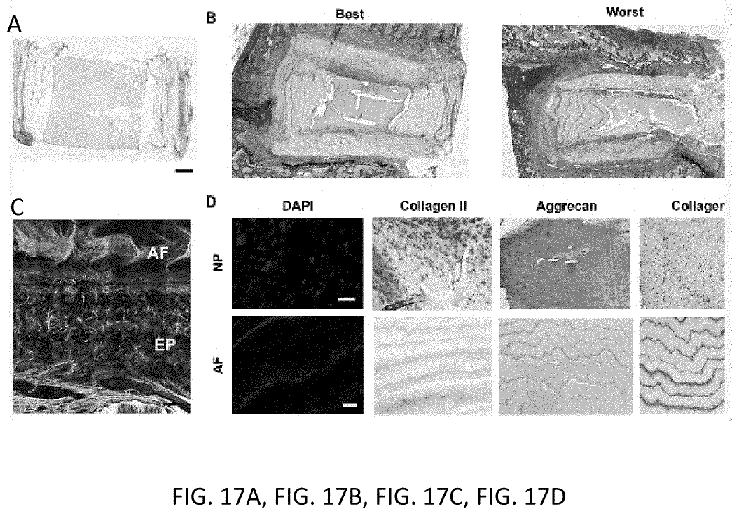

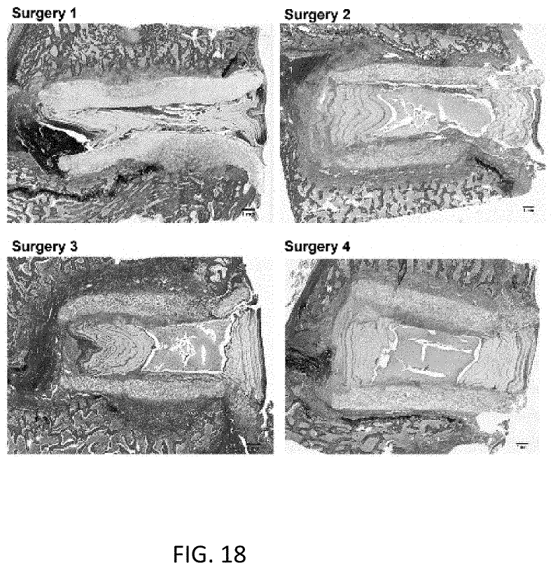

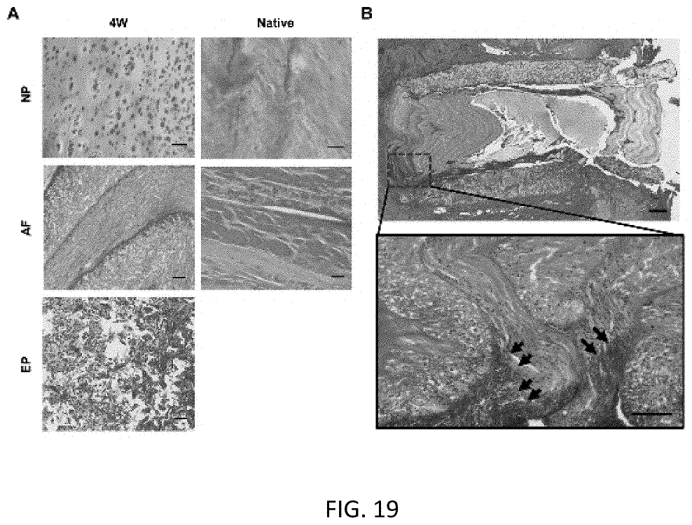

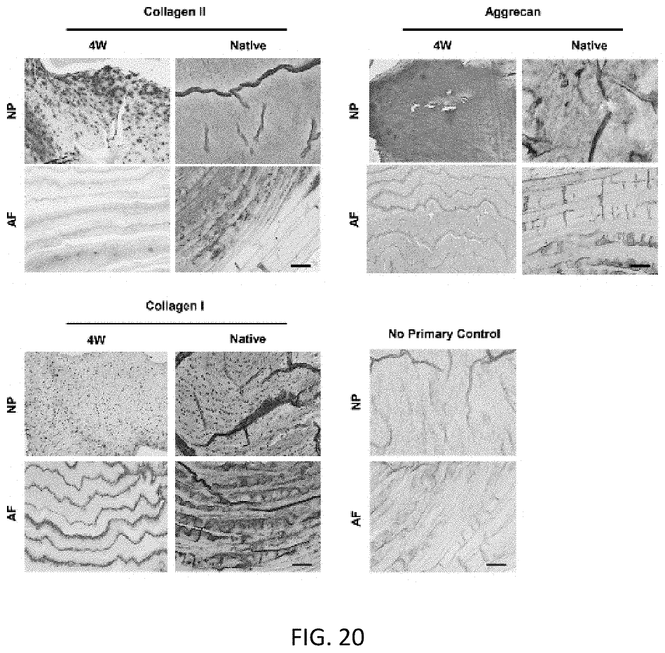

After 4 weeks in vivo, Alcian blue and picrosirius red stained mid-sagittal histology sections demonstrated that eDAPS structure was preserved within the goat cervical disc space, and that matrix distribution and content were maintained or slightly improved compared to pre-implantation values (FIG. 17A-B, FIG. 18). Histology and SHG images also demonstrated nascent integration of the endplate region with the native tissue. SHG signal was present within the PCL foam, and was contiguous with the signal from the adjacent vertebral body and AF region of the eDAPS, indicative of new organized collagen matrix deposition within the initially acellular PCL foam endplates (FIG. 17C). Additionally, cellularity of the NP and AF regions of the eDAPS was maintained over the 4-week implantation period, and there was evidence of endogenous cell infiltration into the PCL foam endplates. (FIG. 17D, FIG. 19). Immunohistochemistry for collagen II, aggrecan, and collagen I demonstrated that the matrix composition of the eDAPS generally recapitulated that characteristic of the native disc, with a collagen II and aggrecan rich NP and an AF composed primarily of collagen I (FIG. 17D, FIG. 20). Hematoxylin and eosin staining revealed some infiltration of neutrophils into the outer layers of the eDAPS AF in three of four animals, indicative of a localized mild inflammatory response, potentially due to the allogenic cell source (FIG. 19). However, this was limited to the outermost region of the implant and animals demonstrated no clinical signs of infection, implant rejection, or functional impairment over the study duration.

The remaining three animals were euthanized 8 weeks after implantation, and the eDAPS implants and native goat cervical motion segments were assayed via quantitative MRI and compressive mechanical testing. T2-weighted MRI of eDAPS after 8 weeks implantation demonstrated the maintenance of eDAPS structure in vivo and increased signal intensity in the NP and AF regions compared to pre-implantation values (FIG. 15A-B). Quantitative T2-mapping of the eDAPS implants demonstrated that NP T2 values after 8 weeks implantation were significantly lower (P=0.04) than native T2 values, but were within the range of native healthy goat cervical discs (FIG. 15C). To assess the function of the eDAPS 8 weeks after implantation, vertebra-eDAPS-vertebra motion segments were isolated after removal of the anterior fixation plate and were subjected to compression testing at physiologic loads. The stress applied to the eDAPS was equivalent to that applied to the average human cervical disc space (20 cycles of compression, 0 to 25N, 0 to 0.084 MPa). Mechanical functionality of a tissue engineered disc in vivo in a large animal model has not been previously reported.

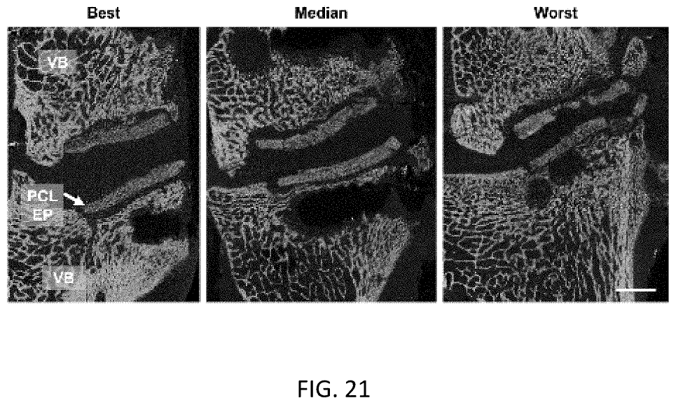

eDAPS compressive mechanical properties increased from their pre-implantation values, and either matched or exceeded the compressive properties of adjacent, native cervical discs (FIG. 15D). The toe region modulus was significantly (P=0.02) increased in eDAPS implanted motion segments compared to pre-implantation values (FIG. 15E), whereas transition and maximum strains were significantly reduced (P=0.04 and P=0.03, respectively) from pre-implantation values after 8 weeks in vivo (FIG. 15F). eDAPS moduli and strains were not significantly different from the native cervical disc after 8 weeks in vivo. This maturation of the mechanical properties of the implants is likely due to progressive integration of the eDAPS with native tissue, as evidenced via μCT imaging (FIG. 21).

Previous studies have pioneered the translation of tissue engineered discs from the rat tail to the beagle cervical spine; however, the beagle cervical disc space is less than half the size of the human cervical disc space. This previous work in the beagle spine found promising results at 4 weeks; however, loss of proteoglycan content and disc height were evident with longer durations, and mechanical properties following implantation were not reported. Herein, a goat cervical disc replacement model was established, which shares similar dimensions to the human cervical spine, and the results demonstrate the feasibility of translation of the eDAPS to this large animal model. eDAPS composition, hydration and cellularity were maintained in vivo, and there was evidence of integration of the eDAPS with the native vertebral bodies. 8 weeks after implantation, the mechanical function of the eDAPS implants was similar to native disc mechanical properties, and demonstrated significant maturation from pre-implantation values.

3. Discussion

Whole disc tissue engineering holds promise as a treatment strategy for patients with end stage disc degeneration and associated spinal pathology necessitating surgical intervention. Upon implantation in vivo, a successful tissue engineered disc replacement would restore native disc space height, integrate with the adjacent vertebral bodies, recapitulate the mechanical function of the disc under physiologic loading, and retain a viable cell population to maintain matrix composition and distribution similar to the native, healthy disc. To progress towards clinical translation, tissue engineered discs can be evaluated using large animal models with comparable geometry, anatomy, and mechanics to the human spine. Tissue engineering of an intervertebral disc for human clinical application has the additional challenge of length scale, with disc heights of 5 mm for the cervical spine and 11 mm for the lumbar spine. The intervertebral disc is also unique in that it is the largest avascular structure in the body, resulting in a low nutrient environment that will also pose a challenge to large-scale tissue engineered constructs.

Given these challenges, the majority of the work in the field thus far has been limited to the in vitro characterization of tissue engineered discs at small size scales (2-3 mm in height and 4-10 mm in diameter). Moreover, very few studies have assessed whole tissue engineered discs in vivo within the spine, and when performed, studies have been limited to small animal models. To advance the clinical translation of a tissue engineered whole disc replacement, tissue engineered discs were developed with and without endplates (DAPS and eDAPS), and these constructs were evaluated in vitro at multiple size scales (up to human cervical disc size), and in the short-term in vivo in a small animal model. In this study, the composition and mechanical function of the eDAPS was evaluated for up to 20 weeks in vivo in a rat tail disc replacement model, and additionally evaluated eDAPS sized for the human cervical spine in a large animal model for up to 8 weeks.

Results from this study show that the eDAPS mature compositionally over time in vivo in the rat tail, achieving mechanical properties that are similar to the native disc at 20 weeks. The eDAPS functionally integrated with the adjacent vertebral bodies, yielding robust mechanical properties in tension. Functional integration of a tissue engineered disc in vivo has not been previously demonstrated, yet this is a critical benchmark for clinical translation. Since the function of the native disc is primarily mechanical in nature, whereby compressive loads on the spine are supported via the development of hydrostatic pressure within the NP which places the AF collagen fibers in tension, the interfaces of the native disc with the adjacent vertebral body are critical for proper mechanical function and are essential to recapitulate in a tissue engineered construct after in vivo implantation. In the eDAPS, improvements in tensile mechanical properties were accompanied by increasing maturation of the eDAPS interfaces, particularly the PCL endplate-vertebral body junction, where infiltrating host cells deposited collagen within the endplates that, over time, began to mineralize and vascularize.

Building on these results in the rat tail, this technology was translated to a larger length scale that would be directly applicable to the human cervical spine, and demonstrate successful total disc replacement with an eDAPS in the goat cervical spine. The eDAPS can be successfully fabricated from bone-marrow derived MSCs, a more clinically relevant cell source for disc tissue engineering compared with AF and NP cells. The goat cervical spine is a particularly attractive pre-clinical model, due to its semi-upright stature and the similar height and width of the disc space to the human cervical spine. eDAPS sized for the goat cervical disc could be used in a total disc replacement in humans, using the same surgical approach and instrumentation used in the goat model. Results from this implantation illustrate that after 4 weeks, matrix distribution was either retained or improved within these large-scale eDAPS, with evidence of integration of the eDAPS with the adjacent vertebral bodies. The MRI results indicate that the composition at 8 weeks is maintained or improved from pre-implantation values in vivo in the goat cervical spine, and that the compressive mechanical properties of the eDAPS implanted motion segments either matched or exceeded those of the native goat cervical disc. Despite differences in fabrication (MSCs versus native disc cells and agarose versus hyaluronic acid hydrogels), the maturation trajectory of the eDAPS in vivo in the goat spine thus far parallels our findings in the rat model, including progressive maturation of mechanical properties, nascent integration of the PCL endplates, and maintained composition at early time points.

4. Materials and Methods

i. Study design.

The objectives of this study were to elucidate the in vivo maturation and mechanical properties of a tissue engineered intervertebral disc with endplates (eDAPS) after implantation in both small (rat caudal spine) and large (goat cervical spine) animal models. eDAPS can compositionally mature, functionally integrate with the native tissue over time, and recapitulate native disc mechanical function in these models. For the small animal studies, eDAPS were implanted in the caudal disc space of male athymic rats with external fixation for 10 (n=5) or 20 weeks (n=9). After motion segment harvest, all specimens were subjected to MRI T2 mapping. Samples were then randomly designated for histology (10w: n=2, 20w: n=2), macroscale mechanical testing (10w: n=4, 20w: n=6), microscale mechanical testing (20w: n=4), and biochemistry (10w: n=3, 20w: n=4). For large animal studies, eDAPS were implanted in the cervical disc space of male large frame goats with internal fixation for 4 (n=4) or 8 weeks (n=3). At 4 weeks, all implanted motion segments were processed for histology. At 8 weeks, all eDAPS implanted motion segments underwent quantitative MRI T2 mapping, and compressive mechanical testing. For both small and large animal studies, the adjacent, native healthy intervertebral discs were utilized as controls. Data were not blinded, and no data were excluded from this study.

ii. Statistical Analysis

Statistical analyses were performed in Prism (Graph Pad Software Inc.), with significance defined as P<0.05. All data are shown as mean±standard deviation. Data were assumed to be non-normally distributed, as sample sizes were too low to test for normality (Shapiro-Wilk normality test). A Kruskal-Wallis test with a Dunn's multiple comparison test was used to assess differences in MRI T2 values, GAG and collagen content, and mechanical properties in tension and compression for eDAPS implanted in the rat tail disc space for 10 and 20 weeks, compared to either native and pre-implantation values. A Kruskal-Wallis test with a Dunn's multiple comparisons was used to assess differences in compressive mechanical properties between goat eDAPS implants before and after 8 weeks implantation, compared to native goat cervical discs. A two-tailed Mann-Whitney test was used to assess statistical differences in strain measured via μCT compression testing between 20 week eDAPS implanted motion segments and native discs, and differences in NP T2 values in 8 week goat eDAPS implants compared to native goat discs.

iii. Cell Isolation and Expansion.

Bovine AF and NP cells were isolated from the caudal discs (˜3 years old and ˜2 hours after sacrifice, JBS Souderton Inc,), as previously described. Allogeneic goat MSCs were isolated from iliac crest bone marrow aspirates from large frame goats (˜3 years of age) taken during surgeries for unrelated projects, as previously described. Bovine disc cells and goat MSCs were expanded to passage 2 in basal media consisting of high glucose Dulbecco's Modified Eagle Medium (DMEM, Gibco, Invitrogen Life Sciences), 10% fetal bovine serum (FBS, Gibco) and 1% penicillin/streptomycin/fungizone (PSF, Gibco).

iv. eDAPS Fabrication and In Vitro Culture for Rat Caudal Spine Implantation.

eDAPS sized for the rat caudal spine (2 mm high, 4-5 mm diameter) were fabricated as previously described. The AF region of the eDAPS was fabricated from concentric layers of electrospun poly (ε-caprolactone) (PCL), where the orientation of nanofibers within each layer alternated at ±30° to the eDAPS long axis to match the structure of the native AF. Intervening layers of poly (ethylene oxide) (PEO) were included between PCL layers, and were subsequently dissolved away upon scaffold hydration and sterilization. The AF scaffolds were coated in 20 μg/mL of fibronectin (Sigma-Aldrich), and bovine AF cells were seeded on the top and bottom side of the AF region (1×10⁶ cells per side), infiltrating between the AF layers. The NP region of the DAPS was fabricated from a methyacrylated hyaluronic acid (MeHA) hydrogel. Bovine NP cells (20 million cells/mL) were suspended in 1% w/v MeHA dissolved in 0.05% photoinitiator (Irgacure 2959, Ciba-Geigy). The MeHA hydrogel was UV cured for 10 minutes between two glass plates and punched to yield gels 2 mm in diameter and 1.5 mm high. PCL foam endplates were fabricated via salt leaching and punched to create acellular constructs 4 mm in diameter and 1.5 mm high, with a pore size of ˜100 μm. In a subset of eDAPS utilized for the 20 week implantation group, zirconia nanoparticles were incorporated within the PCL foams to render them radiopaque.

Following cell seeding, the AF and NP regions of the eDAPS were cultured separately for 2 weeks in a chemically defined media (CM+), consisting of high glucose DMEM supplemented with 1% PSF, 40 ng/mL dexamethasone (Sigma-Aldrich), 50 μg/mL ascorbate 2-phosphate (Sigma-Aldrich), 40 μg/mL L-proline (Sigma-Aldrich), 100 μg/mL sodium pyruvate (Corning Life Sciences), 0.1% insulin, transferrin, and selenious acid (ITS Premix Universal Culture Supplement; Corning), 1.25 mg/mL bovine serum albumin (Sigma-Aldrich), 5.35 μg/mL linoleic acid (Sigma-Aldrich), and 10 ng/mL TGF-β3 (R&D Systems). Media were changed three times per week. After two weeks, the AF and NP regions of the eDAPS were combined, and two acellular PCL foam endplates were apposed to either side of the AF region by passing two 31 G needles through the height of the construct. The eDAPS were cultured for an additional 3 weeks (5 weeks total pre-culture) in CM+ prior to implantation. The needles were removed prior to implantation.

v. eDAPS Fabrication and In Vitro Culture for Goat Cervical Spine Implantation.

To fabricate eDAPS sized for the goat cervical disc space, strips of electrospun, aligned PCL 6 mm in width and 150 mm in length were cut into strips at an angle of 30° to the fiber direction and directly seeded with goat MSCs at a density of 3 million cells per side, as previously described. MSC seeded strips were cultured for 1 week in CM+, after which the AF region was assembled by layering 4 strips to achieve opposing fiber directions)(±30°, and wrapping using a custom mold to create a concentric, lamellar construct with an outer diameter of 16 mm. The AF region was cultured for an additional week in CM+ on an orbital shaker. The NP region was generated by seeding goat MSCs into a 2% agarose hydrogel, as previously described. Agarose was utilized for the goat eDAPS as it is difficult to UV cure the hyaluronic acid hydrogel at the thicknesses required for the goat eDAPS. NP hydrogels were punched to create constructs 8 mm diameter and 6 mm high, which were cultured for 2 weeks in CM+. Acellular porous PCL was fabricated via salt leaching and punched to yield endplates 1.5 mm high and 16 mm diameter. After 2 weeks of culture, the AF and NP regions were combined with the PCL endplates as described above to form the eDAPS. The eDAPS were cultured in 20 mL of CM+ media on an orbital shaker for 13-15 weeks prior to implantation in the goat cervical spine, with media changes 3 times a week. FIG. 6 depicts a schematic of eDAPS fabrication and cell seeding for the rat and goat models.

vi. eDAPS In Vivo Implantation.

eDAPS were implanted in the rat caudal disc space of athymic rats (Foxn1^{mu} retired breeders, Envigo) after 5 weeks of preculture, as previously described. Two kirschner wires were passed through the C8 and C9 vertebral bodies, allowing for the placement of a rigid external fixator designed to immobilize the implanted level. The native disc was removed, and a partial corpectomy of the vertebral bodies adjacent to the disc (˜1-2 mm bone removed per endplate) was performed using a high speed burr. The eDAPS were then placed into the opening, the skin closed with suture, and the rats returned to normal cage activity for the remainder of the study. Animals were euthanized 10 weeks (n=5) or 20 weeks (n=9) post-implantation for analysis. Native rat tail motion segments (C6-C7 level) were obtained from the level above the eDAPS implant and utilized as controls (n=10).

To implant eDAPS in the goat cervical disc space, male large frame goats (˜3 years of age, Thomas D. Morris, Inc.) were positioned in dorsal recumbency under general anesthesia. The C2-C3 disc space was localized with imaging prior to incision. A transverse incision was made on the left side of the cervical spine over the C2-C3 interspace and dissection was carried medial to the carotid sheath through the retropharyngeal space where the spine was palpated. Soft tissues and muscle anterior to the spine were dissected in a subperiosteal manner to expose the lateral extents of the intervertebral space and the adjacent third of the cranial and caudal vertebral bodies. After completion of the surgical exposure the native disc and portion of the adjacent vertebral cartilaginous endplate were removed; distraction was applied to the intervertebral space using a Caspar cervical distractor system to afford access to the dorsal (posterior) third of the interspace. Discectomy and endplate resection (˜1-2 mm bone removed per endplate) was performed utilizing a combination of straight and angled curettes, rongeurs, and a high-speed burr. Suction and a sterile saline flush were used to clear the interspace of bone fragments and blood generated from the endplate resection. The eDAPS was placed into the prepared and distracted interspace. Following eDAPS placement, distraction was released and the implanted motion segment was immobilized via the placement of a ventral CSLP locking plate (DePuy Synthes). Implant position was confirmed with orthogonal fluoroscopy followed by closing the incision in layers. After recovery from anesthesia, animals returned to standard housing consisting of 12×12 ft stalls with ad libitum exercise. Animals' postoperative recovery was monitored with digital radiography and computed tomography. Dorso-ventral and lateral views in the standing, awake animal were obtained bi-weekly to monitor implant status. Animals were euthanized 4 weeks (n=4) or 8 weeks (n=3) post-implantation for analysis.

vii. MRI Scanning and Analysis.

MRI scans of eDAPS-implanted and control rat caudal motion segments were performed using a 4.7T scanner (Magnex Scientific Limited) and a custom-made 17 mm diameter solenoid coil. A multi-echo-multi-spin sequence was used to acquire a series of images for quantitative T2 mapping (0.5 mm slice thickness, 117 μm in plane resolution, 16 echoes, TR/TE=2,000/11.13 ms). Average T2 maps for each experimental group were generated using a custom MATLAB code, as previously described. For eDAPS implanted goat cervical spines, T2-weighted (5 mm slice thickness, 0.5 mm in plane resolution, TR/TE=4,540/123 ms) mid-sagittal images were obtained using a 3T scanner (Siemens Magnetom TrioTim). A series of images of the cervical spine for T2 mapping was also obtained (6 echoes, TE=13 ms, 5 mm slice thickness, 0.5 mm in plane resolution).

viii. Mechanical Testing and Analysis.

Vertebra-eDAPS-vertebra motion segments and native motion segments were prepared for compression testing by carefully removing the skin of the tail and clearing the vertebral bodies adjacent to the eDAPS of soft tissue (with adjacent muscle and tendon left intact). Ink spots were placed on the vertebral bone immediately distal and proximal to the eDAPS to serve as fiducial markers for optical displacement tracking during testing. To determine compressive mechanical properties, motion segments were potted in a low melting temperature indium casting alloy (McMaster-Carr) in custom fixtures, and subjected to a testing protocol consisting of 20 cycles of compression from 0 to −3N (0 to −0.25 MPa) at 0.05 Hz (Instron 5948) in a bath of phosphate buffered saline (PBS) at room temperature. Mechanical properties (toe and linear region modulus, transition and maximum strain) were calculated from the 20th cycle of compression in MATLAB and normalized to disc area and height measured from MR images, as previously described. The compressive mechanical properties of eDAPS cultured in vitro for 5 weeks (pre-implantation) were also quantified in a similar fashion. After macroscale compression testing, motion segments from the 20 week implantation group were subjected to μCT scanning and compression testing as described below.

After compression testing, a complete circumferential dissection of the muscle and tendons surrounding the eDAPS was performed. eDAPS implanted and native motion segments were then subjected to tensile testing to failure at 0.025 mm/sec. A bi-linear fit of the tension curves in MATLAB was used to quantify toe and linear tensile modulus and failure stress and strain.

For eDAPS implanted in the goat cervical disc space for 8 weeks, vertebral body-eDAPS-vertebral body, or native cervical disc motion segments were isolated, and the posterior and lateral boney elements were removed with a hand saw. The cranial and caudal vertebral bodies were potted in a low melting temperature alloy, and specimens were subjected to compressive testing protocol (Instron 5948) in a bath of PBS consisting of 20 cycles of compression from 0 to −25N (0 to 0.084 MPa). A bi-linear fit of the compression curves in MATLAB was performed to quantify toe and linear region modulus, and transition and maximum strains for eDAPS and the native goat cervical disc. eDAPS sized for the goat cervical spine and cultured for 15 weeks were subjected to compressive mechanical testing in a similar fashion to determine the mechanical properties of the eDAPS prior to implantation.

ix. μCT Testing and Analysis.

Native rat tail motion segments and eDAPS implanted motion segments at 20 weeks were subjected to μCT compression testing. Motion segments were wrapped in PBS soaked gauze and placed within the Scanco Medical Compression/Tension Device after potting the proximal and distal portions of the vertebral bodies in paraffin wax to stabilize the motion segment within the device. Motion segments were scanned at 10 μm resolution using a Scanco Medical μCT50 both before and after the application of 3N compressive loading. The height of the native disc, or the disc portion of the eDAPS (excluding the radiopaque PCL foam endplates) was quantified before and after compression using a custom MATLAB code, as previously described. Strain was calculated as the change in disc height with compression divided by the original disc height.

x. Evaluation of eDAPS Composition and Structure.

After mechanical testing, eDAPS were dissected from the motion segment and manually separated into AF, NP and EP portions and individually digested overnight in proteinase K at 60° C. GAG content of each region was determined using the dimethylmethylene blue (DMMB) dye binding assay, and collagen content was quantified via the p-diaminobenzaldehyde/chloramine-T assay for ortho-hydroxyproline (OHP). GAG and collagen content were normalized to sample wet weight, and compared to the biochemical content of eDAPS cultured in vitro for 5 weeks (pre-implantation).

eDAPS implanted rat caudal motion segments (n=2-3 per time point), eDAPS implanted goat cervical motion segments (n=4), and native rat caudal and goat cervical motion segments were fixed, decalcified (Formical-2000, Decal Chemical Corporation, Tallman, N.Y.) and processed through paraffin. 10 μm sections were stained with Alcian blue (glycosaminoglycans) and picrosirius red (collagens). For rat eDAPS, immunohistochemistry was performed for collagen II (DSHB, II-II6B3) and chondroitin sulfate (DSHB, 9BA12) and Collagen I (Millipore, AB749P). For goat eDAPS, immunohistochemistry was performed for collagen II (DSHB, II-II6B3), aggrecan (Millipore, ABT1373), and collagen I (Millipore, AB749P). For each antibody, sections from each experimental group were stained simultaneously. Rehydrated sections were serially incubated at room temperature in proteinase K (Dako) for 5 minutes, 3% hydrogen peroxide for 10 minutes, horse serum for 30 minutes (Vectastain ABC Universal Kit, Vector Laboratories), and primary antibody overnight at 4° C. Secondary visualization was achieved using the Vectastain ABC Universal HRP Kit (PK-6200, Vector Laboratories) and 3,3′-diaminobenzidine (Millipore).

xi. Second Harmonic Generation Imaging.

Paraffin embedded, 10 μm sections of both eDAPS and native discs were mounted on glass slides, cleared with citrisolv, and rehydrated prior to mounting with permount and coverslipping. Sections were viewed on a Nikon AIR confocal microscope equipped with a Specta Physics Deep See Insight tunable laser set to 880 nm for collagen second harmonic generation. Z-stacks of 0.4 μm thickness were captured across the section depth and presented as an average intensity projection.

### C. Example 3 Hydroxyapaptite Coating of Porous Polycaprolactone to Enhance Integration of a Tissue-Engineered Total Disc Replacement

Forming the interfaces between the intervertebral discs of the spine and the adjacent vertebral bodies are the endplates, which consist of a thin layer of hyaline cartilage and an adjacent layer of cortical bone. With aging or following injury, degeneration of the intervertebral discs and adjacent endplates commonly occurs and is frequently associated with back pain. There is a significant need to develop new treatment strategies to address both the disc and vertebral endplate. Towards this end, tissue engineered total disc replacements have been developed with endplates (endplate modified disc like angle-ply structures, eDAPS) for the treatment of severe, advanced-stage disc and endplate degeneration. In contrast to other designs for tissue engineered whole discs, the porous polymer endplate analog of the eDAPS provides an interface through which integration of the engineered disc with the native vertebral body can occur. However, even after 30 weeks implantation in a large animal model, robust mineralization of this interface is still not observed. The purpose of this study is to optimize the design of the endplate region, via the inclusion of a hydroxyapatite (HA) coating, to improve integration of the eDAPS following in vivo implantation.

Scaffold Fabrication and HA coating: Porous poly(ε-caprolactone) (PCL) foams were fabricated via a salt leaching method to generate constructs 4 mm in diameter and 1.5 mm thick. To coat the PCL foams in HA, foams were hydrated through a gradient of ethanol, followed by serial overnight immersions in and 2M NaOH and simulated body fluid (SBF).

In Vitro Studies: Prior to cell seeding, PCL only foams and HA coated PCL foams were hydrated and sterilized through an ethanol gradient and coated overnight in fibronectin. P2 bovine bone-marrow derived mesenchymal stem cells (MSCs) were seeded on the top and bottom surface of each foam at a density of 3,333 cells/mm². MSC-seeded foams were cultured in either basal or osteogenic media (n=4 per group) for 5 weeks. At the end of the culture duration, construct viability (MTT assay) and alkaline phosphatase activity (ALP, Sigma Aldrich kit) were quantified. Additional samples (n=3 per group) were cryosectioned in the sagittal plane and stained for calcium deposits using a Von Kossa stain kit (Abcam).

In Vivo Studies: For in vivo evaluation of the HA coating, PCL foams 4 mm in diameter and 5 mm thick were fabricated, to mimic the size of the eDAPS constructs. The tail disc spaces of five athymic rats were implanted with acellular PCL foams (n=2) or HA-coated PCL foams (n=3), in a surgical procedure and with an external fixator. Briefly, the native C8-C9 tail disc space was removed, and a partial corpectomy of the adjacent vertebral bodies was performed with a high-speed burr such that the constructs could be placed in apposition with the marrow of the vertebral bodies. After 10 weeks, the animals were euthanized and vertebral body-PCL foam-vertebral body motion segments harvested for analysis. Motion segments were fixed in formalin and subjected to μCT scanning at 10 μm resolution to visualize the three-dimensional tissue distribution within the PCL foam following in vivo implantation. Samples were then decalcified and processed for paraffin histology. Histologic sections were stained with the Mallory-Heidenhain trichrome stain to distinguish unmineralized collagen (light grey) from mineralized collagen (dark gray), and immunohistochemistry (IHC) was performed for osteocalcin. Significant differences (p<0.05) between groups were assessed via an ANOVA with Tukey's post-hoc test

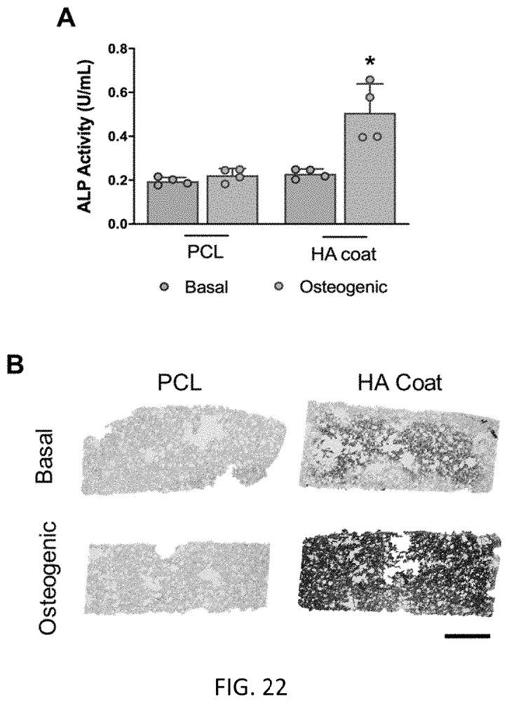

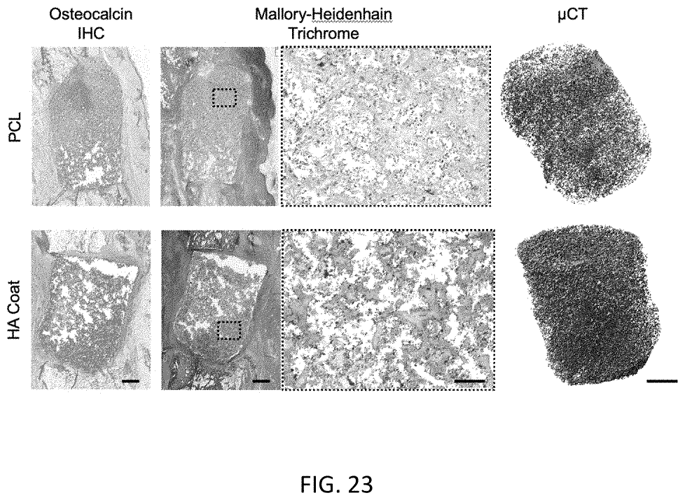

In vitro studies of HA-coated and PCL only constructs seeded with bone marrow-derived MSCs demonstrated a significant increase in construct ALP activity in the HA-coated group cultured in osteogenic media (FIG. 22A). There were no statistically significant differences in MTT absorbance across groups. Von Kossa staining the constructs showed increased calcium deposition in the HA coated group compared to the PCL only group, in both basal and osteogenic media culture conditions (FIG. 22B). In vivo, collagenous matrix deposition occurred within the initially acellular constructs in both the PCL only and HA coated groups after 10 weeks implantation. There was increased immunohistochemical staining for osteocalcin present in the HA coated group compared to the PCL only controls (FIG. 23, left panel). Additionally, the Mallory-Heidenhain trichrome stain demonstrated areas of mineralized collagen (pink staining) present in the HA coated groups which were not present in the PCL only controls (FIG. 23, middle panels). 3D μCT reconstructions of the constructs 10 weeks post-implantation further demonstrated increased tissue deposition in the HA coated group compared to the PCL only control (FIG. 23, right panel).

The results from the in vitro and in vivo experiments indicate that coating of porous PCL foams with HA can increase their osteogenic potential. In previous work where eDAPS with PCL only endplates were implanted in the rat caudal disc space, mineralized collagen was not observed within the endplate region until 20 weeks post-implantation. Here, staining for mineralized collagen was observed within the construct at 10 weeks post-implantation in the HA coated group, indicating that integration can be accelerated by the HA coating. These findings are consistent with previous work in the fracture healing field, where hydroxyapatite coating by other methods improved in vitro and in vivo osteogenesis.

These results show that the design modifications to the tissue engineered endplate and intervertebral disc replacement can improve and accelerate integration of the construct with the native bone, which can be critical for clinical translation.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. R. D. Bowles, H. H. Gebhard, R. Härtl, L. J. Bonassar, Tissue-engineered intervertebral discs produce new matrix, maintain disc height, and restore biomechanical function to the rodent spine., Proc. Natl. Acad. Sci. U S. A. 108, 13106-11 (2011).
2. Y. Moriguchi, R. Navarro, P. Grunert, J. Mojica, K. Hudson, T. Khair, M. Alimi, L. Bonassar, R. Hartl, Total Disc Replacement Using Tissue Engineered Intervertebral Discs In An In-vivo Beagle Model, PLoS One 12, 1-18 (2017).
3. H. Mizuno, A. K. Roy, C. a Vacanti, K. Kojima, M. Ueda, L. J. Bonassar, Tissue-engineered composites of anulus fibrosus and nucleus pulposus for intervertebral disc replacement., Spine (Phila. Pa. 1976). 29, 1290-1297; discussion 1297-1298 (2004).
4. H. Mizuno, A. K. Roy, V. Zaporojan, C. a Vacanti, M. Ueda, L. J. Bonassar, Biomechanical and biochemical characterization of composite tissue-engineered intervertebral discs., Biomaterials 27, 362-70 (2006).
5. L. J. Nesti, W.-J. J. Li, R. M. Shanti, Y. J. Jiang, W. Jackson, B. A. Freedman, T. R. Kuklo, J. R. Giuliani, R. S. Tuan, Intervertebral disc tissue engineering using a novel hyaluronic acid-nanofibrous scaffold (HANFS) amalgam., Tissue Eng Part A 14, 1527-1537 (2008).
6. Y. Zhuang, B. Huang, C. Q. Li, L. T. Liu, Y. Pan, W. J. Zheng, G. Luo, Y. Zhou, Construction of tissue-engineered composite intervertebral disc and preliminary morphological and biochemical evaluation, Biochem. Biophys. Res. Commun. 407, 327-332 (2011).
7. M. Lazebnik, S. M, P. Glatt, L. Friis, C. Berkland, M. Detamore, Biomimetic method for combining the nucleus pulposus and annulus fibrosus, J. Tissue Eng. Regen. Med. 5, e179-e187 (2011).
8. S.-H. Park, E. S. Gil, H. Cho, B. B. Mandal, L. W. Tien, B.-H. Min, D. L. Kaplan, Intervertebral disk tissue engineering using biphasic silk composite scaffolds., Tissue Eng. Part A 18, 447-58 (2012).
9. T. K. Chik, W. H. Chooi, Y. Y. Li, F. C. Ho, H. W. Cheng, T. H. Choy, K. Y. Sze, K. K. D. Luk, K. M. C. Cheung, B. P. Chan, Bioengineering a multicomponent spinal motion segment construct-A 3D model for complex tissue engineering, Adv. Healthc. Mater. (2014), doi:10.1002/adhm.201400192.
10. B. Xu, H. Xu, Y. Wu, X. Li, Y. Zhang, X. Ma, Q. Yang, Intervertebral disc tissue engineering with natural extracellular matrix-derived biphasic composite scaffolds, PLoS One 10, 1-16 (2015).
11. J. Iu, E. Massicotte, S.-Q. Li, M. B. Hurtig, E. Toyserkani, J. P. Santerre, R. Kandel, In Vitro Generated Intervertebral Discs: Towards Engineering Tissue Integration, Tissue Eng. Part A 23, 1001-1010 (2017).
12. S. E. Gullbrand, D. H. Kim, E. Bonnevie, B. G. Ashinsky, L. J. Smith, D. M. Elliott, R. L. Mauck, H. E. Smith, Towards the Scale up of Tissue Engineered Intervertebral Discs for Clinical Application, Acta Biomater. 70, 154-164 (2018).
13. J. T. Martin, S. E. Gullbrand, B. G. Mohanraj, B. G. Ashinsky, D. H. Kim, K. Ikuta, D. M. Elliott, L. J. Smith, R. L. Mauck, H. E. Smith, Optimization of pre-culture conditions to maximize the in vivo performance of cell-seeded engineered intervertebral discs, Tissue Eng. Part A 23, 923-234 (2017).
14. J. T. Martin, S. E. Gullbrand, D. Kim, K. Ikuta, C. G. Pfeifer, B. G. Ashinsky, L. J. Smith, D. M. Elliott, H. E. Smith, R. L. Mauck, In Vitro Maturation and In Vivo Integration and Function of an Engineered Cell-Seeded Disc-like Angle Ply Structure (DAPS) for Total Disc Arthroplasty, Sci. Rep. 7, 15765 (2017).
15. S. E. Gullbrand, B. G. Ashinsky, E. D. Bonnevie, D. H. Kim, J. B. Engiles, L. J. Smith, D. M. Elliott, T. P. Schaer, H. E. Smith, R. L. Mauck, Long-term mechanical function and integration of an implanted tissue-engineered intervertebral disc, Sci. Transl. Med. 10, eaau0670 (2018).
1. R. A. Deyo, S. K. Mirza, B. I. Martin, Back pain prevalence and visit rates: Estimates from U.S. national surveys, 2002, Spine (Phila. Pa. 1976). 31, 2724-2727 (2006).
2. C. J. L. Murray, T. Vos, R. Lozano, Disability-adjusted life years (DALYs) for 291 diseases and injuries in 21 regions, 1990-2010: a systematic analysis for the Global Burden of Disease Study 2010., Lancet 380, 2197-223 (2012).
3. B. Martin, R. A. DEYO, S. Mirza, J. Turner, B. Comstock, W. Hollingworth, S. Sullivan, Expenditures and health status among adults with back and neck problems, JAMA 299, 656-664 (2008).
4. American Academy of Orthopaedic Surgeons and the United States Bone and Joint Decade, The Burden of Musculoskeletal Diseases in the United States (American Academy of Orthopaedic Surgeons, Rosemont, Ill., 2008).
5. J. P. G. Urban, S. Roberts, Degeneration of the intervertebral disc, Arthritis Res. Ther. 5, 120-130 (2003).
6. M. Haefeli, F. Kalberer, D. Saegesser, A. G. Nerlich, N. Boos, G. Paesold, The course of macroscopic degeneration in the human lumbar intervertebral disc., Spine (Phila. Pa. 1976). 31, 1522-1531 (2006).
7. G. Ghiselli, J. C. Wang, N. N. Bhatia, W. K. Hsu, E. G. Dawson, Adjacent segment degeneration in the lumbar spine., J. Bone Joint Surg. Am. 86-A, 1497-503 (2004).
8. B. J. C. Freeman, J. Davenport, Total disc replacement in the lumbar spine: A systematic review of the literature, Eur. Spine J. 15 (2006), doi:10.1007/s00586-006-0186-9.
9. W. Jacobs, A. Tuschel, M. de Kleuver, W. Peul, A. Verbout, C. Oner, Total disc replacement for chronic low-back pain, Cochrane Database Syst. Rev. 38, 24-36 (2010).
10. A. J. Buckland, J. F. Baker, R. P. Roach, J. M. Spivak, Cervical disc replacement—emerging equivalency to anterior cervical discectomy and fusion, Int. Orthop. 40, 1329-1334 (2016).
11. S. M. Kurtz, A. van Ooij, R. Ross, J. de Waal Malefijt, J. Peloza, L. Ciccarelli, M. L. Villarraga, Polyethylene wear and rim fracture in total disc arthroplasty, Spine J. 7, 12-21 (2007).
12. H. Mizuno, A. K. Roy, V. Zaporojan, C. a Vacanti, M. Ueda, L. J. Bonassar, Biomechanical and biochemical characterization of composite tissue-engineered intervertebral discs., Biomaterials 27, 362-70 (2006).
13. N. L. Nerurkar, S. Sen, A. H. Huang, D. M. Elliott, R. L. Mauck, Engineered disc-like angle-ply structures for intervertebral disc replacement., Spine (Phila. Pa. 1976). 35, 867-73 (2010).
14. M. Lazebnik, S. M, P. Glatt, L. Friis, C. Berkland, M. Detamore, Biomimetic method for combining the nucleus pulposus and annulus fibrosus, J. Tissue Eng. Regen. Med. 5, e179-e187 (2011).
15. K. D. Hudson, R. I. Mozia, L. J. Bonassar, Dose-Dependent Response of Tissue-Engineered Intervertebral Discs to Dynamic Unconfined Compressive Loading., Tissue Eng. Part A 21, 564-572 (2015).
16. J. T. Martin, S. E. Gullbrand, B. G. Mohanraj, B. G. Ashinsky, D. H. Kim, K. Ikuta, D. M. Elliott, L. J. Smith, R. L. Mauck, H. E. Smith, Optimization of pre-culture conditions to maximize the in vivo performance of cell-seeded engineered intervertebral discs, Tissue Eng. Part A 23, 923-234 (2017).
17. B. Xu, H. Xu, Y. Wu, X. Li, Y. Zhang, X. Ma, Q. Yang, Intervertebral disc tissue engineering with natural extracellular matrix-derived biphasic composite scaffolds, PLoS One 10, 1-16 (2015).
18. J. lu, E. Massicotte, S.-Q. Li, M. B. Hurtig, E. Toyserkani, J. P. Santerre, R. Kandel, In Vitro Generated Intervertebral Discs: Towards Engineering Tissue Integration, Tissue Eng. Part A 23, 1001-1010 (2017).
19. R. D. Bowles, H. H. Gebhard, R. Hartl, L. J. Bonassar, Tissue-engineered intervertebral discs produce new matrix, maintain disc height, and restore biomechanical function to the rodent spine., Proc. Natl. Acad. Sci. U.S.A 108, 13106-11 (2011).
20. Y. Moriguchi, R. Navarro, P. Grunert, J. Mojica, K. Hudson, T. Khair, M. Alimi, L. Bonassar, R. Hartl, Total Disc Replacement Using Tissue Engineered Intervertebral Discs In An In-vivo Beagle Model, PLoS One 12, 1-18 (2017).
21. J. T. Martin, S. E. Gullbrand, D. Kim, K. Ikuta, C. G. Pfeifer, B. G. Ashinsky, L. J. Smith, D. M. Elliott, H. E. Smith, R. L. Mauck, In Vitro Maturation and In Vivo Integration and Function of an Engineered Cell-Seeded Disc-like Angle Ply Structure (DAPS) for Total Disc Arthroplasty, Sci. Rep. 7, 15765 (2017).
22. J. T. Martin, A. H. Milby, J. a Chiaro, D. H. Kim, N. M. Hebela, L. J. Smith, D. M. Elliott, R. L. Mauck, Translation of an engineered nanofibrous disc-like angle-ply structure for intervertebral disc replacement in a small animal model., Acta Biomater. 10, 2473-81 (2014).
23. N. L. Nerurkar, B. M. Baker, S. Sen, E. E. Wible, D. M. Elliott, R. L. Mauck, Nanofibrous biologic laminates replicate the form and function of the annulus fibrosus., Nat. Mater. 8, 986-92 (2009).
24. N. L. Nerurkar, D. M. Elliott, R. L. Mauck, Mechanical design criteria for intervertebral disc tissue engineering., J. Biomech. 43, 1017-30 (2010).
25. R. D. Bowles, L. A. Setton, Biomaterials for intervertebral disc regeneration, Biomaterials 129, 54-67 (2017).
26. C. W. a. Pfirrmann, A. Metzdorf, M. Zanetti, J. Hodler, N. Boos, Magnetic Resonance Classification of Lumbar Intervertebral Disc Degeneration, Spine (Phila. Pa. 1976). 26, 1873-1878 (2001).
27. S. E. Gullbrand, B. G. Ashinsky, J. T. Martin, S. Pickup, L. J. Smith, R. L. Mauck, H. E. Smith, Correlations between quantitative T2 and Tlp MRI, mechanical properties and biochemical composition in a rabbit lumbar intervertebral disc degeneration model, J. Orthop. Res. 34, 1382-8 (2016).
28. D. W. Hukins, A simple model for the function of proteoglycans and collagen in the response to compression of the intervertebral disc., Proceedings. Biol. Sci. 249, 281-285 (1992).
29. E. D. Bonnevie, R. L. Mauck, Physiology and Engineering of the Graded Interfaces of Musculoskeletal Junctions, Annu. Rev. Biomed. Eng. 20, 403-429 (2018).
30. J. C. Beckstein, S. Sen, T. P. Schaer, E. J. Vresilovic, D. M. Elliott, Comparison of animal discs used in disc research to human lumbar disc: axial compression mechanics and glycosaminoglycan content., Spine (Phila. Pa. 1976). 33, E166-73 (2008).
31. M. Cannella, A. Arthur, S. Allen, M. Keane, A. Joshi, E. Vresilovic, M. Marcolongo, The role of the nucleus pulposus in neutral zone human lumbar intervertebral disc mechanics, J. Biomech. 41, 2104-2111 (2008).
32. J. I. Boxberger, J. D. Auerbach, S. Sounok, D. M. Elliott, An in vivo model of reduced nucleus pulposus glycosaminoglycan content in the rat lumbar intervertebral disc, Spine (Phila. Pa. 1976). 33, 146-154 (2009).
33. J. E. Cason, A rapid one-step Mallory-Heidenhain stain for connective tissue., Stain Technol. 25, 225-226 (1950).
34. G. D. O. Connell, E. J. Vresilovic, D. M. Elliott, Comparison of Animals Used in Disc Research to Human Lumbar Disc Geometry, 32, 328-333 (2007).
35. D. Coric, R. D. Guyer, P. D. Nunley, D. Musante, C. Carmody, C. Gordon, C. Lauryssen, M. O. Boltes, D. D. Ohnmeiss, Prospective, randomized multicenter study of cervical arthroplasty versus anterior cervical discectomy and fusion: 5-year results with a metal-on-metal artificial disc, J. Neurosurg. Spine 28, 1-10 (2018).
36. S. Reitmaier, F. Graichen, A. Shirazi-Adl, H. Schmidt, Separate the Sheep from the Goats, J. Bone Jt. Surg. 99, e102 (2017).
37. S. E. Gullbrand, D. H. Kim, E. Bonnevie, B. G. Ashinsky, L. J. Smith, D. M. Elliott, R. L. Mauck, H. E. Smith, Towards the Scale up of Tissue Engineered Intervertebral Discs for Clinical Application, Acta Biomater. 70, 154-164 (2018).
38. I. Busscher, J. J. W. Ploegmakers, G. J. Verkerke, A. G. Veldhuizen, Comparative anatomical dimensions of the complete human and porcine spine., Eur. Spine J. 19, 1104-14 (2010).
39. J. P. G. Urban, S. Smith, J. C. T. Fairbank, Nutrition of the intervertebral disc., Spine (Phila. Pa. 1976). 29, 2700-2709 (2004).
40. L. J. Nesti, W.-J. J. Li, R. M. Shanti, Y. J. Jiang, W. Jackson, B. A. Freedman, T. R. Kuklo, J. R. Giuliani, R. S. Tuan, Intervertebral disc tissue engineering using a novel hyaluronic acid-nanofibrous scaffold (HANFS) amalgam., Tissue Eng Part A 14, 1527-1537 (2008).
41. S.-H. Park, E. S. Gil, H. Cho, B. B. Mandal, L. W. Tien, B.-H. Min, D. L. Kaplan, Intervertebral disk tissue engineering using biphasic silk composite scaffolds., Tissue Eng. Part A 18, 447-58 (2012).
42. K. D. Hudson, L. J. Bonassar, Hypoxic Expansion of Human Mesenchymal Stem Cells Enhances 3D Maturation of Tissue Engineered Intervertebral Discs, Tissue Eng. Part A 23, ten.TEA.2016.0270 (2016).
43. M. D. Humzah, R. W. Soames, Human intervertebral disc: structure and function. Anat. Rec. 220, 337-56 (1988).
44. D. W. Hukins, A simple model for the function of proteoglycans and collagen in the response to compression of the intervertebral disc., Proc. R. Soc. B Biol. Sci. 249, 281-5 (1992).
45. B. Berg-Johansen, A. J. Fields, E. C. Liebenberg, A. Li, J. C. Lotz, Structure-function relationships at the human spinal disc-vertebra interface, J. Orthop. Res. 36, 192-201 (2018).
46. J. Qin, X. He, D. Wang, P. Qi, L. Guo, S. Huang, X. Cai, H. Li, R. Wang, Artificial Cervical Vertebra and Intervertebral Complex Replacement through the Anterior Approach in Animal Model: A Biomechanical and In Vivo Evaluation of a Successful Goat Model, PLoS One 7, e52910 (2012).
47. I. E. Erickson, A. H. Huang, C. Chung, R. T. Li, J. A. Burdick, R. L. Mauck, Differential Maturation and Structure-Function Relationships in Mesenchymal Stem Cell- and Chondrocyte-Seeded Hydrogels, Tissue Eng. Part A 15, 1041-1052 (2009).
48. J. A. Mojica-Santiago, G. M. Lang, R. Navarro-Ramirez, I. Hussain, R. Hard, L. J. Bonassar, Resorbable Plating System Stabilizes Tissue-Engineered Intervertebral Discs Implanted Ex-Vivo in Canine Cervical Spines, JOR Spine, e1031 (2018).
49. S. E. Gullbrand, T. P. Schaer, P. Agarwal, J. R. Bendigo, G. R. Dodge, W. Chen, D. M. Elliott, R. L. Mauck, N. R. Malhotra, L. J. Smith, Translation of an injectable triple-interpenetrating-network hydrogel for intervertebral disc regeneration in a goat model, Acta Biomater. 60, 201-209 (2017).
50. B. Johnstone, T. M. Hering, a I. Caplan, V. M. Goldberg, J. U. Yoo, In vitro chondrogenesis of bone marrow-derived mesenchymal progenitor cells., Exp. Cell Res. 238, 265-272 (1998).
51. J. T. Martin, C. M. Collins, K. Ikuta, R. L. Mauck, D. M. Elliott, Y. Zhang, D. G. Anderson, A. R. Vaccaro, T. J. Albert, V. Arlet, H. E. Smith, Population average T2 MRI maps reveal quantitative regional transformations in the degenerating rabbit intervertebral disc that vary by lumbar level, J. Orthop. Res. 33, 140-148 (2015).
52. J. I. Boxberger, J. D. Auerbach, S. Sen, D. M. Elliott, An In Vivo Model of Reduced Nucleus Pulposus Glycosaminoglycan Content in the Rat Lumbar Intervertebral Disc, Spine (Phila. Pa. 1976). 33, 146-154 (2008).
53. J. T. Martin, D. J. Gorth, E. E. Beattie, B. D. Harfe, L. J. Smith, D. M. Elliott, Needle puncture injury causes acute and long-term mechanical deficiency in a mouse model of intervertebral disc degeneration, J. Orthop. Res. 31, 1276-1282 (2013).

1. An engineered vertebral disc implant comprising:
an engineered vertebral disc, wherein the engineered vertebral disc comprises a nucleus pulposus region and an annulus fibrosus region; and
two endplates, wherein the endplates comprise a porous polymer foam, and wherein the endplates comprise channels.

2. The engineered vertebral disc implant of claim 1 wherein the endplates further comprise hydroxyapatite.

3. (canceled)

4. (canceled)

5. The engineered vertebral disc implant of claim 1, wherein the endplates have a bone interface side and a vertebral disc interface side, wherein at least one of the two endplates comprises:
a body; and
a projection on the bone interface side that extends outwardly from the body.

6. (canceled)

7. The engineered vertebral disc implant of claim 1, wherein at least one of the two endplates further comprises one or more vascular-promoting agents.

8. The engineered vertebral disc implant of claim 7, wherein the one or more vascular-promoting agents are vascular endothelial growth factor, deferoxamine, nimodipine, or phthalimide neovascularization factor (PNF-1).

9. The engineered vertebral disc implant of claim 1, wherein the nucleus pulposus region comprises a top surface, bottom surface, and a side edge extending between the top and bottom surfaces and defining a perimeter of the nucleus pulposus region, wherein the perimeter of the nucleus pulposus is circumferentially surrounded by the annulus fibrosus region.

10. (canceled)

11. The engineered vertebral disc implant of claim 5, wherein the annulus fibrosus region comprises a top surface, a bottom surface, an inner side edge, and an outer side edge that defines a perimeter of the annulus fibrosus region, wherein the inner side edge and the outer side edge extend between the top surface and the bottom surface, wherein the vertebral disc interface side of a first endplate is attached to the top surfaces of the nucleus pulposus and annulus fibrosus regions, and wherein the vertebral disc interface side of a second endplate is attached to the bottom surfaces of the nucleus pulposus and annulus fibrosus regions.

12. (canceled)

13. The engineered vertebral disc implant of claim 1, wherein the nucleus pulposus region comprises a hydrogel.

14. The engineered vertebral disc implant of claim 13, wherein the hydrogel comprises viable cells.

15. (canceled)

16. The engineered vertebral disc implant of claim 13, wherein the hydrogel is a hyaluronic acid or agarose hydrogel.

17. The engineered vertebral disc implant of claim 1, wherein the annulus fibrosus region comprises one or more sheets of nano-fibrous PCL.

18. (canceled)

19. The engineered vertebral disc implant of claim 1, wherein the annulus fibrosus region further comprises polyethylene oxide (PEO).

20. The engineered vertebral disc implant of claim 1, wherein the annulus fibrosus region comprises viable cells.

21. (canceled)

22. (canceled)

23. The engineered vertebral disc implant of claim 14, wherein the cells of the nucleus pulposus region or annulus fibrosus region have been previously cultured.

24.-38. (canceled)

39. The engineered vertebral disc implant of claim 1, wherein the porous polymer foam is poly (ε-caprolactone) (PCL), poly(lactic-co-glycolic acid), polylactic acid, poly-DL-lactide, or polydiaxanone.

40. A method of treating disc degeneration comprising implanting one or more of the engineered vertebral disc implants of claim 1 to a subject in need thereof, wherein the endplates of the engineered vertebral disc implant are attached to the vertebra of the subject.

41. The method of claim 40, wherein the engineered vertebral disc implant is cultured prior to implanting to a patient.

42. (canceled)

43. (canceled)

44. The method of claim 40, further comprising removing the degenerated disc prior to implanting the engineered vertebral disc implant.

45. The method of claim 40, further comprising removing a portion of the vertebra prior to implanting engineered vertebral disc implants.

46. The method of claim 45, wherein removing a portion of the vertebra comprises scraping or drilling into the vertebra.

47. The method of claim 40, wherein the cells in the engineered vertebral disc implant are the cells obtained from the subject.

48. The method of claim 40, wherein the endplates are attached to the vertebra via the bone interface side.

49.-52. (canceled)

53. The engineered vertebral disc implant of claim 2, wherein at least one of the two endplates further comprises one or more vascular-promoting agents.

54. The method of claim 40, wherein the endplates further comprise hydroxyapatite.

55. The method of claim 54, wherein at least one of the two endplates further comprises one or more vascular-promoting agents.
